(19) United States
(12) Patent Application Publication
(10) Pub. No.: US 2015/0320852 A1
(43) Pub. Date: Nov. 12, 2015

(54) CONJUGATES FOR PROTECTING AGAINST DIPHTHERIA AND/OR TETANUS

(71) Applicant: GLAXOSMITHKLINE BIOLOGICALS SA, Rixensart (BE)

(72) Inventor: Michael BROEKER, Marburg (DE)

(21) Appl. No.: 14/652,234

(22) PCT Filed: Dec. 16, 2013
(86) PCT No.: PCT/EP2013/076781

§ 371 Date: Jun. 15, 2015

Related U.S. Application Data
(60) Provisional application No. 61/738,958, filed on Dec. 18, 2012.

(51) Int. Cl. A61K 39/095; A61K 39/05; A61K 39/102; A61K 39/13; A61K 39/02; A61K 39/29; C12N 7/00; A61K 39/08; A61K 39/09

(52) U.S. Cl. CPC A61K 39/095; A61K 39/08; A61K 39/05; A61K 39/102; A61K 39/092; A61K 39/099; A61K 39/292; C12N 7/00; A61K 39/13; A61K 2039/55

(57) ABSTRACT

Saccharide conjugate vaccines which use diphtheria toxoid or tetanus toxoid as a carrier protein can confer protection against lethal challenge by diphtheria toxin or tetanus toxin. Thus, in addition to protecting against the bacteria whose saccharides have been attached to the carrier, such conjugate vaccines can also be used to protect against diphtheria and tetanus, so the diphtheria toxoid and tetanus toxoid components of current complex combination vaccines may be superfluous. Therefore the antigenic complexity of these vaccines can be reduced without reducing their breadth of protection, and removing these superfluous components creates space in the vaccine for adding immunogens for protecting against further pathogens. The same effect is not seen with a CRM197 carrier, but this observation makes this carrier more attractive for conjugate vaccines which are given concomitantly with infant combination vaccines that contain Dt and Tt.

This application claims the benefit of U.S. provisional application 61/738,958 (filed 18 Dec. 2012), the complete contents of which are hereby incorporated herein by reference for all purposes.

### TECHNICAL FIELD

This invention is in the field of immunisation, in particular using conjugate vaccines.

### BACKGROUND ART

Vaccines containing antigens from more than one pathogenic organism within a single dose are known as “combination” vaccines. Various combination vaccines have been approved, including early trivalent vaccines for protecting against diphtheria, tetanus and pertussis (“DTP” vaccines). The most complex multi-pathogen vaccines currently available are 6-valent and include antigens for diphtheria, tetanus, pertussis, polio, hepatitis B and Hib (D-T-aP-IPV-HBV-Hib). These vaccines are already very complex and gaining approval for vaccines with further antigens is not straightforward.

The 6-valent vaccines include Hib saccharide which is conjugated to a tetanus toxoid carrier protein. Known conjugate vaccines against other pathogens include the MENVEO™ and PREVNAR™ products for meningococcus and pneumococcus, respectively. After receiving conjugate vaccines it is known that antibodies are raised not only against the saccharide but also against the carrier protein. Typical carrier proteins include diphtheria and tetanus toxoids. These are themselves protective antigens, but reference 1 reports that conjugates of these toxoids are “not sufficient to induce complete immunity with respect to the carrier”. Possible explanations why conjugation removes the toxoids' protective efficacy could be that protective epitopes (linear or conformational) are destroyed or masked by the covalent coupling of saccharide, or that conjugation reduces flexibility of the carrier protein.

Despite this general loss of protective efficacy caused by conjugation, reference 1 reports that tetanus or diphtheria toxoids can retain their protective effects even after conjugation of Streptococcus pneumoniae saccharides. The author did not extrapolate that finding to any other saccharides, but did expect that the same result would be seen with CRM197 (see [0041] in ref. 1), which is a non-toxic mutant of diphtheria toxin. CRM197 is another well-known carrier protein in vaccine saccharide conjugates, and it differs from diphtheria toxin by a single amino acid mutation.

Reference 2 reports a study of a 4-valent meningococcal conjugate vaccine (now approved as the NIMENRIX™ product) using a tetanus toxoid. The author reports that 100% of vaccine recipients raised anti-tetanus antibodies, but these patients would already have received routine pediatric vaccines that include tetanus toxoid, and the proportion of patients with anti-tetanus antibodies before receiving the 4-valent meningococcal vaccine was already more than 90%. Thus reference 2 does not give any information about whether the conjugate vaccine could induce a significant anti-tetanus immune response in un-primed naïve infants. Moreover, reference 2 detected anti-tetanus antibodies using an ELISA test which cannot reveal whether those antibodies are protective. Other tests for measuring such antibodies (e.g. the CHO neutralisation assay used to determine the neutralising effect of anti-diphtheria antibodies elicited by the MENACTRA™ product) also do not reveal whether the antibodies are protective in vivo.

CRM197 has also been studied in this way. Reference 3 showed that CRM197 is immunogenic in humans but, again, the immune response was measured adults who had previously received diphtheria toxoid vaccines, rather than in naïve patients, and the immune response was determined by an in vitro assay (ELISA) rather than a functional assay.

Although diphtheria and tetanus toxoids retain at least some immunogenicity after being conjugated to bacterial saccharides, it is therefore unclear whether they retain their protective efficacy. Thus it is unknown whether vaccines such as NIMENRIX™ or MENACTRA™ can elicit protective anti-tetanus or anti-diphtheria immunity in immunologically naïve subjects. Similarly, it is unclear whether conjugated CRM197, as used in the MENVEO™ and PREVNAR™ products, can elicit protective anti-diphtheria immunity in these subjects.

### SUMMARY OF THE INVENTION

The inventor has shown that existing saccharide conjugate vaccines which use diphtheria toxoid or tetanus toxoid as a carrier protein (such as the MENACTRA™ and MENITORIX™ products), but do not contain the toxoid as a separate antigen, can confer protection against lethal challenge by diphtheria toxin or tetanus toxin. Thus, in addition to protecting against the bacteria whose saccharides have been attached to the carrier, such conjugate vaccines can also be used to protect against diphtheria and tetanus. This means that the diphtheria and tetanus toxoid components of current complex combination vaccines may be superfluous. Therefore the antigenic complexity of these vaccines can be reduced without reducing their breadth of protection. Furthermore, removing these superfluous components creates space in the vaccine for adding immunogens for protecting against further pathogens. For example, an existing hexavalent vaccine D-T-P-HBV-IPV-Hib could (a) be simplified by removing the unconjugated T component and relying on a T carrier in the Hib conjugate, (b) be expanded without increasing antigenic complexity by replacing the unconjugated T component with a MenC conjugate having a T carrier, and/or (c) be greatly expanded, without a corresponding increase in antigenic complexity, by replacing the unconjugated D component with MenACWY-D conjugates and using a T carrier in the Hib conjugate in place of unconjugated T.

Thus a first aspect of the invention provides a method for immunising an infant against multiple pathogens, comprising a step of co-immunising the infant with: (a) a vaccine containing unconjugated diphtheria toxoid, but not containing unconjugated tetanus toxoid; and (b) a vaccine containing a saccharide conjugated to a tetanus toxoid carrier.

A second aspect of the invention provides a method for immunising an infant against multiple pathogens, comprising a step of co-immunising the infant with: (a) a vaccine containing unconjugated tetanus toxoid, but not containing unconjugated diphtheria toxoid; and (b) a vaccine containing a saccharide conjugated to a diphtheria toxoid carrier.

A third aspect of the invention provides a method for immunising an infant against multiple pathogens, comprising a step of co-immunising the infant with: (a) a vaccine which is free from unconjugated tetanus toxoid and is free from unconjugated diphtheria toxoid; (b) a vaccine containing a saccharide conjugated to a tetanus toxoid carrier; and (c) a vaccine containing a saccharide conjugated to a diphtheria toxoid carrier.

A fourth aspect of the invention provides a combination vaccine comprising unconjugated diphtheria toxoid, and a saccharide conjugated to a tetanus toxoid carrier, but being free from unconjugated tetanus toxoid.

A fifth aspect of the invention provides a combination vaccine comprising unconjugated tetanus toxoid, and a saccharide conjugated to a diphtheria toxoid carrier, but being free from unconjugated diphtheria toxoid.

A sixth aspect of the invention provides a combination vaccine comprising a saccharide conjugated to a tetanus toxoid carrier, and a saccharide conjugated to a diphtheria toxoid carrier, but being free from unconjugated tetanus toxoid and free from unconjugated diphtheria toxoid.

A seventh aspect of the invention provides a kit comprising at least two kit components which, when mixed, result in the combination vaccine of the third to sixth aspects.

An eighth aspect of the invention provides a method for immunising an infant against meningococcal disease and tetanus, comprising a step of administering a vaccine containing a meningococcal capsular saccharide conjugated to a tetanus toxoid carrier, without administering unconjugated tetanus toxoid.

A ninth aspect of the invention provides a method for immunising an infant against meningococcal disease and diphtheria, comprising a step of administering a vaccine containing a meningococcal capsular saccharide conjugated to a diphtheria toxoid carrier, without administering unconjugated diphtheria toxoid.

Although diphtheria and tetanus conjugates can confer protection against lethal challenge by diphtheria toxin or tetanus toxin, the inventor has shown that the same effect is not seen with a CRM197 carrier (which differs from diphtheria toxin by a single amino acid mutation). Thus CRM197-based conjugates cannot be used in the way discussed above, but the inventor's discovery has a different impact. As CRM197 is a weaker diphtheria immunogen than Dt in the context of a conjugate vaccine, it is more attractive as a carrier when a conjugate vaccine is given concomitantly with current infant combination vaccines (which contain Dt and Tt) because they can offer a lower potential for negative interference induced by the carrier protein. Thus a tenth aspect of the invention provides a method for immunising an infant against multiple pathogens, comprising a step of co-immunising the infant with (a) a vaccine containing diphtheria toxoid and tetanus toxoid; and one of (b1) a vaccine containing a meningococcal capsular saccharide conjugated to a CRM197 carrier; (b2) a vaccine containing a pneumococcal capsular saccharide conjugated to a CRM197 carrier; (b3) a first vaccine containing a meningococcal capsular saccharide conjugated to a CRM197 carrier and a second vaccine containing a pneumococcal capsular saccharide conjugated to a CRM197 carrier; or (b4) a vaccine containing pneumococcal and meningococcal capsular saccharides, each conjugated to CRM197 carriers.

The Infant

The invention is used to immunise infants i.e. human beings from birth up to the age of 12 months e.g. between 0-9 months, or 0-6 months. Thus, for instance, the infant may be aged 2 months, 3 months, 4 months, 5 months, or 6 months.

The invention is particularly useful in connection with an infant's first immunisation against diphtheria and tetanus, which typically takes place at the age of 2 months. Thus the infant is ideally immunologically naïve to tetanus toxoid (Tt) and/or diphtheria toxoid (Dt) at the time of immunisation.

Co-Immunisation Where the invention refers to co-immunisation, the different vaccines in an enumerated list can be administered either separately or as a combination.

Where the vaccines are administered separately, they will typically be administered at different sites e.g. one vaccine to the left upper arm, and a second vaccine to the right upper arm. Thus two vaccines may be administered contralaterally (e.g. both arms, or both legs, or a contralateral arm and leg) or ipsilaterally (e.g. the arm and leg on the same side of the body). Although the vaccines are administered separately, they are administered at substantially the same time (e.g. during the same medical consultation or visit to a healthcare professional or vaccination centre), such as within 1 hour of each other.

Rather than co-immunising separately, however, administration as a combination is preferred. Thus a preferred co-immunisation uses a combination vaccine i.e. a single composition in which the different immunogens are admixed. Combination vaccines offer patients the advantage of receiving a reduced number of injections, which can lead to the clinical advantage of increased compliance (e.g. see chapter 29 of ref. 4), particularly in pediatric patients. In the invention's first aspect, for instance, the infant preferably receives a single composition which includes the unconjugated Dt and the saccharide conjugated to a Tt carrier.

Conjugated and Unconjugated Toxoid Carriers

Where a toxoid is a conjugated toxoid, it is covalently linked (directly or via a linker) to another moiety, which will typically be a saccharide antigen (e.g. a bacterial capsular saccharide).

The 1st-9th aspects of the invention refer to vaccines which include or administer (or do not include/administer) “unconjugated” Dt and/or Tt. This term means that the toxoid has not been conjugated to another antigen e.g. to a saccharide antigen. Thus, for example, an “unconjugated” Tt would exclude the Tt which is present in the conjugated PRP-T or NIMENRIX™ products, and an “unconjugated” Dt would exclude the Dt which is present in the conjugated PRP-D or MENACTRA™ products.

When a vaccine is defined as containing a particular unconjugated toxoid, it can also (unless explicitly specified) include that same toxoid in conjugated form e.g. a vaccine including unconjugated Tt could include both Tt and PRP-T. Conversely, when a vaccine is defined as not containing (or as being free from) a particular unconjugated toxoid, it can (and usually will) include that toxoid in conjugated form e.g. a vaccine which does not contain unconjugated Tt could nevertheless include Hib-T.

The term “unconjugated” in relation to a toxoid does not refer to toxoid which was used to prepare a conjugate but which, for whatever reason, has remained as unreacted residual toxoid or which has become deconjugated. Thus, for instance, is a conjugation reaction involving a toxoid and a saccharide is incomplete then a small residual amount of unreacted toxoid could remain (even after purification), and this will be carried through into compositions if the conjugate is then mixed with other components. Similarly, if a conjugate is stored for a long period of time, or is stored under harsh conditions, breakdown can occur such that deconjugation occurs. When a composition is said not to contain an unconjugated toxoid, it can nevertheless include post-conjugation residual or deconjugated toxoid if this was present in a conjugated toxoid component which was used when making the composition. The skilled person can recognise the difference between unconjugated toxoid which is present on purpose, and toxoid which is instead present as a residual contaminant or as a breakdown product, so will readily understand when a composition is indeed free from unconjugated toxoid. For instance, the invention relates to human vaccines which are tightly-regulated products made by well-defined processes, and a skilled person making a composition which contains a conjugated toxoid but is free from that toxoid in unconjugated form will not use a component in which that toxoid has never been subjected to a conjugation reaction; conversely, a skilled person making a composition which contains an unconjugated toxoid will not use a toxoid which was previously subjected to a conjugation reaction. Thus, when a vaccine is defined as not containing an unconjugated toxoid, any post-conjugation residual or deconjugated forms of that toxoid will make up <10% by weight of the total amount of that toxoid in the vaccine (e.g. <5%, <2%, or <1%).

Where a vaccine is intended to protect against tetanus, it will include enough immunogenic tetanus toxoid to meet the European Pharmacopoeia requirements for tetanus vaccination (protection of mice against lethal challenge by tetanus toxin). Similarly, where a vaccine is intended to protect against diphtheria, it will include enough immunogenic diphtheria toxoid to meet the European Pharmacopoeia requirements for diphtheria vaccination (protection of guinea pigs against lethal challenge by diphtheria toxin).

Vaccines with Unconjugated Diphtheria Toxoid, but No Unconjugated Tetanus Toxoid

The first aspect of the invention co-immunises with: (a) a vaccine containing unconjugated Dt, but not containing unconjugated Tt; and (b) a vaccine containing a saccharide conjugated to a Tt carrier.

When the co-immunisation with (a) and (b) occurs as a combination vaccine, this gives the fourth aspect of the invention.

Thus these vaccines are not made using unconjugated Tt, and to protect against tetanus they instead include a saccharide conjugated to a Tt carrier. Such conjugated saccharides with a Tt carrier include, but are not limited to: a meningococcal saccharide, such as the conjugates present in any of the NEISVAC-C™, MENHIBRIX™, MENITORIX™ or NIMENRIX™ products; a pneumococcal saccharide, such as the serotype 18C conjugate present in the SYNFLORIX™ product; a H. influenzae type B saccharide, such as the conjugate present in any of the HIBTITER™, MENHIBRIX™, MENITORIX™ or HIBERIX™ products.

Thus the invention can use one or more of the following saccharides, conjugated to a Tt carrier: a meningococcal serogroup A capsular saccharide; a meningococcal serogroup C capsular saccharide; a meningococcal serogroup W135 capsular saccharide; a meningococcal serogroup X capsular saccharide; a meningococcal serogroup Y capsular saccharide; a pneumococcal serotype 18C capsular saccharide; a Salmonella enterica serovar Typhi (S. Typhi) virulence capsular polysaccharide (‘Vi’); and/or a H. influenzae type B capsular saccharide.

In addition, the vaccine can include further saccharide(s) which are conjugated to non-Tt carrier(s) e.g. any of the other 10 conjugates present within the SYNFLORIX™ product. If a vaccine does not include Tt-conjugated capsular saccharides from meningococcal serogroups A, C, W135 & Y, it can include these as CRM197-conjugated saccharides as in the MENVEO™ product, or as Dt-conjugated saccharides as in the MENACTRA™ product. If a vaccine does not include Tt-conjugated capsular saccharides from pneumococcus, it can include these as CRM197-conjugated saccharides from the PREVNAR™ or PREVNAR13™ products. If a vaccine does not include Tt-conjugated Vi capsular saccharides from S. Typhi, it can include this as a Dt-conjugated or CRM197-conjugated saccharide [5,6].

Specific examples of vaccines (a) and (b) which may be used to co-immunise infants within the first aspect of the invention, and of combination vaccines of the fourth aspect of the invention, include but are not limited to:

| (a) | (b) | Combination |
|---|---|---|
| Dt + aP | Hib-Tt | Dt + aP + Hib-Tt |
| Dt + aP + HBsAg | Hib-Tt | Dt + aP + HBsAg + Hib-Tt |
| Dt + aP + IPV | Hib-Tt | Dt + aP + IPV + Hib-Tt |
| Dt + aP + HBsAg + IPV | Hib-Tt | Dt + aP + HBsAg + IPV + Hib-Tt |
| Dt + aP + Hib-CRM197 | MenC-Tt | Dt + aP + Hib-CRM197 + MenC-Tt |
| Dt + aP + HBsAg + Hib-CRM197 | MenC-Tt | Dt + aP + HBsAg + Hib-CRM197 + MenC-Tt |
| Dt + aP + IPV + Hib-CRM197 | MenC-Tt | Dt + aP + IPV + Hib-CRM197 + MenC-Tt |
| Dt + aP + HBsAg + IPV + Hib- | MenC-Tt | Dt + aP + HBsAg + IPV + Hib-CRM197 + MenC-Tt |
| CRM197 | | |
| Dt + aP | Hib-Tt + MenC-Tt | Dt + aP + Hib-Tt + MenC-Tt |
| Dt + aP + HBsAg | Hib-Tt + MenC-Tt | Dt + aP + HBsAg + Hib-Tt + MenC-Tt |
| Dt + aP + IPV | Hib-Tt + MenC-Tt | Dt + aP + IPV + Hib-Tt + MenC-Tt |
| Dt + aP + HBsAg + IPV | Hib-Tt + MenC-Tt | Dt + aP + HBsAg + IPV + Hib-Tt + MenC-Tt |
| Dt + aP | Hib-Tt + MenC-Tt + MenY-Tt | Dt + aP + Hib-Tt + MenC-Tt + MenY-Tt |
| Dt + aP + HBsAg | Hib-Tt + MenC-Tt + MenY-Tt | Dt + aP + HBsAg + Hib-Tt + MenC-Tt + MenY-Tt |
| Dt + aP + IPV | Hib-Tt + MenC-Tt + MenY-Tt | Dt + aP + IPV + Hib-Tt + MenC-Tt + MenY-Tt |
| Dt + aP + HBsAg + IPV | Hib-Tt + MenC-Tt + MenY-Tt | Dt + aP + HBsAg + IPV + Hib-Tt + MenC-Tt + MenY-Tt |
| Dt + aP | Hib-Tt + MenACWY-Tt | Dt + aP + Hib-Tt + MenACWY-Tt |
| Dt + aP + HBsAg | Hib-Tt + MenACWY-Tt | Dt + aP + HBsAg + Hib-Tt + MenACWY-Tt |
| Dt + aP + IPV | Hib-Tt + MenACWY-Tt | Dt + aP + IPV + Hib-Tt + MenACWY-Tt |
| Dt + aP + HBsAg + IPV | Hib-Tt + MenACWY-Tt | Dt + aP + HBsAg + IPV + Hib-Tt + MenACWY-Tt |
| Dt + aP + Hib-CRM197 | MenACWY-Tt | Dt + aP + Hib-CRM197 + MenACWY-Tt |
| Dt + aP + HBsAg + Hib-CRM197 | MenACWY-Tt | Dt + aP + HBsAg + Hib-CRM197 + MenACWY-Tt |
| Dt + aP + IPV + Hib-CRM197 | MenACWY-Tt | Dt + aP + IPV + Hib-CRM197 + MenACWY-Tt |
| Dt + aP + HBsAg + IPV + Hib- | MenACWY-Tt | Dt + aP + HBsAg + IPV + Hib-CRM197 + MenACWY-Tt |
| CRM197 | | |
| Dt + aP + Hib-Tt | MenC-Tt | Dt + aP + Hib-Tt + MenC-Tt |
| Dt + aP + HBsAg + Hib-Tt | MenC-Tt | Dt + aP + HBsAg + Hib-Tt + MenC-Tt |
| Dt + aP + IPV + Hib-Tt | MenC-Tt | Dt + aP + IPV + Hib-Tt + MenC-Tt |
| Dt + aP + HBsAg + IPV + Hib-Tt | MenC-Tt | Dt + aP + HBsAg + IPV + Hib-Tt + MenC-Tt |
| Dt + aP + Hib-Tt | MenX-Tt | Dt + aP + Hib-Tt + MenX-Tt |
| Dt + aP + HBsAg + Hib-Tt | MenX-Tt | Dt + aP + HBsAg + Hib-Tt + MenX-Tt |
| Dt + aP + IPV + Hib-Tt | MenX-Tt | Dt + aP + IPV + Hib-Tt + MenX-Tt |
| Dt + aP + HBsAg + IPV + Hib-Tt | MenX-Tt | Dt + aP + HBsAg + IPV + Hib-Tt + MenX-Tt |
| Dt + aP + Hib-Tt | MenACWY-Tt | Dt + aP + Hib-Tt + MenACWY-Tt |
| Dt + aP + HBsAg + Hib-Tt | MenACWY-Tt | Dt + aP + HBsAg + Hib-Tt + MenACWY-Tt |
| Dt + aP + IPV + Hib-Tt | MenACWY-Tt | Dt + aP + IPV + Hib-Tt + MenACWY-Tt |
| Dt + aP + HBsAg + IPV + Hib-Tt | MenACWY-Tt | Dt + aP + HBsAg + IPV + Hib-Tt + MenACWY-Tt |
| Dt + aP | Vi-Tt | Dt + aP + Vi-Tt |
| Dt + aP + HBsAg | Vi-Tt | Dt + aP + HBsAg + Vi-Tt |
| Dt + aP + IPV | VI-Tt | Dt + aP + IPV + Vi-Tt |
| Dt + aP + HBsAg + IPV | Vi-Tt | Dt + aP + HBsAg + IPV + Vi-Tt |
| Dt + aP + Hib-Tt | Vi-Tt | Dt + aP + Vi-Tt + Hib-Tt |
| Dt + aP + HBsAg + Hib-Tt | Vi-Tt | Dt + aP + HBsAg + Vi-Tt + Hib-Tt |
| Dt + aP + IPV + Hib-Tt | VI-Tt | Dt + aP + IPV + Vi-Tt + Hib-Tt |
| Dt + aP + HBsAg + IPV + Hib-Tt | Vi-Tt | Dt + aP + HBsAg + IPV + Vi-Tt + Hib-Tt |

Six particularly preferred combination vaccines of the fourth aspect are: (a) Dt, aP, HBsAg, IPV, Hib-Tt; (b) Dt, aP, HBsAg, IPV, Hib-Tt, MenC-Tt; (c) Dt, aP, HBsAg, IPV, Hib-Tt, MenC-CRM197; (d) Dt, aP, HBsAg, IPV, Hib-Tt, MenACWY-CRM197; (e) Dt, aP, HBsAg, IPV, Hib-Tt, MenACWY-Dt; (f) Dt, aP, HBsAg, IPV, Hib-Tt, MenACWY-Tt; (g) Dt, aP, HBsAg, IPV, Hib-Tt, MenX-Tt; and (h) Dt, aP, HBsAg, IPV, Hib-Tt, MenX-CRM197.

The eighth aspect of the invention provides methods for immunising an infant against meningococcal disease and tetanus, comprising a step of administering a vaccine containing a meningococcal capsular saccharide conjugated to a tetanus toxoid carrier, without administering tetanus toxoid in unconjugated form. Thus the conjugate is used for immunisation against both meningococcus and tetanus, without separately needing the toxoid as an unconjugated immunogen. In addition to the conjugate, the vaccine used with the eighth aspect may include further antigens as detailed here for the first aspect of the invention. Thus the vaccine can protect against more than just meningococcus and tetanus.

Vaccines with Unconjugated Tetanus Toxoid, but No Unconjugated Diphtheria Toxoid

The second aspect of the invention co-immunises with: (a) a vaccine containing unconjugated Tt, but not containing unconjugated Dt; and (b) a vaccine containing a saccharide conjugated to a Dt carrier. When the co-immunisation with (a) and (b) occurs as a combination vaccine, this gives the fifth aspect of the invention.

Thus these vaccines are not made using unconjugated Dt, and to protect against diphtheria they instead include a saccharide conjugated to a Dt carrier. Such conjugated saccharides with a Dt carrier include, but are not limited to: a meningococcal saccharide, such as the conjugates present in the MENACTRA™ product; a pneumococcal saccharide, such as the serotype 19F conjugate present in the SYNFLORIX™ product; a H. influenzae type B saccharide, such as the conjugate present in the PROHIBIT™ product.

Thus the invention can use one or more of the following saccharides, conjugated to a Dt carrier: a meningococcal serogroup A capsular saccharide; a meningococcal serogroup C capsular saccharide; a meningococcal serogroup W135 capsular saccharide; a meningococcal serogroup X capsular saccharide; a meningococcal serogroup Y capsular saccharide; a pneumococcal serotype 19F capsular saccharide; a Vi saccharide; and/or a H. influenzae type B capsular saccharide.

In addition, the vaccine can include further saccharide(s) which are conjugated to non-Dt carrier(s) e.g. any of the other 10 conjugates present within the SYNFLORIX™ product. If a vaccine does not include Dt-conjugated capsular saccharides from meningococcal serogroups A, C, W135 & Y, it can include these as CRM197-conjugated saccharides as in the MENVEO™ product, or as Tt-conjugated saccharides as in the NIMENRIX™ product. If a vaccine does not include Dt-conjugated capsular saccharides from pneumococcus, it can include these as CRM197-conjugated saccharides from the PREVNAR™ or PREVNAR13™ products. If a vaccine does not include Dt-conjugated Vi capsular saccharides from S. Typhi, it can include this as a Tt-conjugated or CRM197-conjugated saccharide.

Specific examples of vaccines (a) and (b) which may be used to co-immunise infants within the second aspect of the invention, and of combination vaccines of the fifth aspect of the invention, include but are not limited to:

| (a) | (b) | Combination |
|---|---|---|
| Tt + aP | Hib-Dt | Tt + aP + Hib-Dt |
| Tt + aP + HBsAg | Hib-Dt | Tt + aP + HBsAg + Hib-Dt |
| Tt + aP + IPV | Hib-Dt | Tt + aP + IPV + Hib-Dt |
| Tt + aP + HBsAg + IPV | Hib-Dt | Tt + aP + HBsAg + IPV + Hib-Dt |
| Tt + aP | Hib-Dt + MenACWY-Dt | Tt + aP + Hib-Dt + MenACWY-Dt |
| Tt + aP + HBsAg | Hib-Dt + MenACWY-Dt | Tt + aP + HBsAg + Hib-Dt + MenACWY-Dt |
| Tt + aP + IPV | Hib-Dt + MenACWY-Dt | Tt + aP + IPV + Hib-Dt + MenACWY-Dt |
| Tt + aP + HBsAg + IPV | Hib-Dt + MenACWY-Dt | Tt + aP + HBsAg + IPV + Hib-Dt + MenACWY-Dt |
| Tt + aP + Hib-CRM197 | MenACWY-Dt | Tt + aP + Hib-CRM197 + MenACWY-Dt |
| Tt + aP + HBsAg + Hib-CRM197 | MenACWY-Dt | Tt + aP + HBsAg + Hib-CRM197 + MenACWY-Dt |
| Tt + aP + IPV + Hib-CRM197 | MenACWY-Dt | Tt + aP + IPV + Hib-CRM197 + MenACWY-Dt |
| Tt + aP + HBsAg + IPV + Hib- | MenACWY-Dt | Tt + aP + HBsAg + IPV + Hib-CRM197 + MenACWY-Dt |
| CRM197 | | |
| Tt + aP + Hib-Tt | MenACWY-Dt | Tt + aP + Hib-Tt + MenACWY-Dt |
| Tt + aP + HBsAg + Hib-Tt | MenACWY-Dt | Tt + aP + HBsAg + Hib-Tt + MenACWY-Dt |
| Tt + aP + IPV + Hib-Tt | MenACWY-Dt | Tt + aP + IPV + Hib-Tt + MenACWY-Dt |
| Tt + aP + HBsAg + IPV + Hib-Tt | MenACWY-Dt | Tt + aP + HBsAg + IPV + Hib-Tt + MenACWY-Dt |

Three particularly preferred combination vaccines of the fifth aspect are: (a) Tt, aP, HBsAg, IPV, Hib-Dt, MenC-CRM197; (b) Tt, aP, HBsAg, IPV, Hib-Tt, MenACWY-Dt; (c) Tt, aP, HBsAg, IPV, Hib-CRM197, MenACWY-Dt.

The ninth aspect of the invention provides methods for immunising an infant against meningococcal disease and diphtheria, comprising a step of administering a vaccine containing a meningococcal capsular saccharide conjugated to a diphtheria toxoid carrier, without administering diphtheria toxoid in unconjugated form. Thus the conjugate is used for immunisation against both meningococcus and diphtheria, without separately needing the toxoid as an unconjugated immunogen. In addition to the conjugate, the vaccine used with the ninth aspect may include further antigens as detailed here for the second aspect of the invention. Thus the vaccine can protect against more than just meningococcus and diphtheria.

Vaccines with No Unconjugated Tetanus or Diphtheria Toxoids

The third aspect of the invention co-immunises with: (a) a vaccine which is free from unconjugated Tt and is free from unconjugated Dt; (b) a vaccine containing a saccharide conjugated to a Tt carrier; and (c) a vaccine containing a saccharide conjugated to a Dt carrier. When the co-immunisation with (a), (b) and (c) occurs as a combination vaccine, this gives the sixth aspect of the invention.

Thus these vaccines are not made using unconjugated Tt or Dt, and to protect against tetanus and diphtheria they instead include a saccharide conjugated to a Dt carrier and a saccharide conjugated to a Dt carrier and. Examples of products containing such saccharide conjugates are discussed above.

Thus the invention can use one or more of the following saccharides, conjugated to Tt or Dt carriers: a meningococcal serogroup A capsular saccharide; a meningococcal serogroup C capsular saccharide; a meningococcal serogroup W135 capsular saccharide; a meningococcal serogroup Y capsular saccharide; a pneumococcal serotype 18C capsular saccharide; a pneumococcal serotype 19F capsular saccharide; and/or a H. influenzae type B capsular saccharide.

In addition, the vaccine can include further saccharide(s) which are conjugated to non-Tt and non-Dt carrier(s) e.g. any of the other 8 pneumococcal saccharides within the SYNFLORIX™ product which are conjugated to protein D, any of the CRM197-conjugated pneumococcal saccharides within the PREVNAR™ or PREVNAR13™ products, and/or any of the CRM197-conjugated meningococcal saccharides within the MENVEO™ product.

Specific examples of vaccines (a) to (c) which may be used to co-immunise infants within the third aspect of the invention, and of combination vaccines of the sixth aspect of the invention, include but are not limited to:

| (a) | (b) | (c) | Combination |
|---|---|---|---|
| aP + HBsAg | Hib-Tt | MenACWY-Dt | aP + HBsAg + Hib-Tt + MenACWY-Dt |
| aP + IPV | Hib-Tt | MenACWY-Dt | aP + IPV + Hib-Tt + MenACWY-Dt |
| aP + HBsAg + IPV | Hib-Tt | MenACWY-Dt | aP + HBsAg + IPV + Hib-Tt + MenACWY-Dt |
| aP + HBsAg | MenC-Tt | Hib-Dt | aP + HBsAg + MenC-Tt + Hib-Dt |
| aP + IPV | MenC-Tt | Hib-Dt | aP + IPV + MenC-Tt + Hib-Dt |
| aP + HBsAg + IPV | MenC-Tt | Hib-Dt | aP + HBsAg + IPV + MenC-Tt + Hib-Dt |
| aP + HBsAg | MenACWY-Tt | Hib-Dt | aP + HBsAg + MenACWY-Tt + Hib-Dt |
| aP + IPV | MenACWY-Tt | Hib-Dt | aP + IPV + MenACWY-Tt + Hib-Dt |
| aP + HBsAg + IPV | MenACWY-Tt | Hib-Dt | aP + HBsAg + IPV + MenACWY-Tt + Hib-Dt |

Further Antigens

Compositions of the invention as defined above include (i) unconjugated diphtheria toxoid and conjugated tetanus toxoid; (ii) unconjugated tetanus toxoid and conjugated diphtheria toxoid; or (iii) conjugated diphtheria toxoid and conjugated tetanus toxoid. These toxoids protect against diphtheria and tetanus, and also against the pathogens from which any conjugated saccharides are derived (e.g. Hib, meningococcal serogroups A/C/W135/Y, various pneumococcal serotypes). In addition to these diphtheria and tetanus toxoids (and conjugated saccharides) the vaccines will include further immunogens for protecting against further pathogens. Thus, for instance, the vaccines can include one or more of: an acellular pertussis (aP) component; a hepatitis B virus surface antigen (HBsAg); an inactivated poliovirus (IPV); a rabies virus immunogen (e.g. as described in chapter 27 of reference 7), which will generally be an inactivated rabies virus virion; a typhoid fever component, such as a Vi saccharide; and/or a yellow fever virus immunogen, such as an inactivated virus prepared from cell culture e.g. from the 17D strain [8].

Preferred combination vaccines of the invention can protect against:
Diphtheria, tetanus, pertussis, poliomyelitis, and disease caused by Hib.
Diphtheria, tetanus, pertussis, poliomyelitis, hepatitis B virus, and disease caused by Hib.
Diphtheria, tetanus, pertussis, poliomyelitis, disease caused by Hib, diseases caused by N. meningitidis serogroup C.
Diphtheria, tetanus, pertussis, poliomyelitis, hepatitis B virus, disease caused by Hib, diseases caused by N. meningitidis serogroup C.
Diphtheria, tetanus, pertussis, poliomyelitis, disease caused by Hib, diseases caused by N. meningitidis serogroups A, C, W135 & Y.
Diphtheria, tetanus, pertussis, poliomyelitis, hepatitis B virus, disease caused by Hib, diseases caused by N. meningitidis serogroups A, C, W135 & Y.
Diphtheria, tetanus, pertussis, poliomyelitis, hepatitis B virus, and disease caused by Hib, disease caused by S. pneumoniae (at least serotypes 4, 6B, 9V, 14, 18C, 19F & 23F; preferably also 1, 5 & 7F; and more preferably also 3, 6A & 19A).
Diphtheria, tetanus, pertussis, poliomyelitis, disease caused by Hib, diseases caused by N. meningitidis serogroup C, disease caused by S. pneumoniae (at least serotypes 4, 6B, 9V, 14, 18C, 19F & 23F; preferably also 1, 5 & 7F; and more preferably also 3, 6A & 19A).
Diphtheria, tetanus, pertussis, poliomyelitis, hepatitis B virus, disease caused by Hib, diseases caused by N. meningitidis serogroup C, disease caused by S. pneumoniae (at least serotypes 4, 6B, 9V, 14, 18C, 19F & 23F; preferably also 1, 5 & 7F; and more preferably also 3, 6A & 19A).
Diphtheria, tetanus, pertussis, poliomyelitis, disease caused by Hib, diseases caused by N. meningitidis serogroups A, C, W135 & Y, disease caused by S. pneumoniae (at least serotypes 4, 6B, 9V, 14, 18C, 19F & 23F; preferably also 1, 5 & 7F; and more preferably also 3, 6A & 19A).
Diphtheria, tetanus, pertussis, poliomyelitis, hepatitis B virus, disease caused by Hib, diseases caused by N. meningitidis serogroups A, C, W135 & Y, disease caused by S. pneumoniae (at least serotypes 4, 6B, 9V, 14, 18C, 19F & 23F; preferably also 1, 5 & 7F; and more preferably also 3, 6A & 19A).

The immunogenic components of these vaccines can be limited to those for protecting against the pathogens listed above, or the vaccines can include further immunogens for further pathogens.

These vaccines can also be given in conjunction rotavirus vaccine, influenza virus vaccine, tick-borne encephalitis vaccine, rabies vaccine, yellow fever vaccine, typhoid fever vaccine, MenX vaccine, etc.

For any given saccharide which is present in conjugated form in a vaccine, it is preferred to include it attached only to one carrier e.g. if MenA (i.e. serogroup A of N. meningitidis) saccharide is included, it would be present as only one of MenA-CRM197, MenA-Dt, or MenA-Tt. Overall, though, if a vaccine includes multiple different saccharides as conjugates, these can be attached to one type of carrier (e.g. Dt or Tt), or to more than one type (e.g. Dt and/or Tt; and optionally CRM).

Kits

The seventh aspect of the invention provides a kit comprising whose kit components can be mixed to give a combination vaccine of the invention.

Thus, although a vaccine can be administered to a patient as a combination, it does not need to be distributed or stored as a combination. For instance, although full-liquid vaccines are known (i.e. where all antigenic components are in aqueous solution or suspension), it is also known to divide immunogens so that they can be mixed extemporaneously at the time/point of use for administration. Such embodiments include liquid/liquid mixing and liquid/solid mixing e.g. by mixing aqueous material with lyophilised material. For instance, in one embodiment a vaccine can be made by mixing: (a) a first component comprising aqueous antigens; and (b) a second component comprising lyophilized antigens. Where a lyophilized kit component is used, this frequently contains conjugated antigens. For instance, a kit might have (a) a liquid component including Dt+aP+HBsAg+IPV; and (b) a lyophilised component including Hib-Tt+MenC-Tt+MenY-Tt.

The two components are preferably in separate containers (e.g. vials and/or syringes), and the invention provides a kit comprising these components (a) and (b).

Vaccines which Contain Both of Unconjugated Diphtheria and Tetanus Toxoids

In contrast to the first nine aspects of the invention, the tenth aspect of the invention uses a vaccine containing both unconjugated diphtheria toxoid and unconjugated tetanus toxoid. The infant is co-immunised with meningococcal and/or pneumococcal capsular saccharide(s) which are conjugated to CRM197 carrier(s).

Where the infant receives a CRM197-conjugated meningococcal capsular saccharide, it is preferred that they do not also receive a Dt-conjugated meningococcal capsular saccharide or a Tt-conjugated meningococcal capsular saccharide.

Where the infant receives a CRM197-conjugated pneumococcal capsular saccharide, it is preferred that they do not also receive a Dt-conjugated pneumococcal capsular saccharide or a Tt-conjugated pneumococcal capsular saccharide.

Where the infant receives both a CRM197-conjugated meningococcal capsular saccharide and a CRM197-conjugated pneumococcal capsular saccharide, it is preferred that they do not also receive any of: a Dt-conjugated meningococcal capsular saccharide; a Tt-conjugated meningococcal capsular saccharide; a Dt-conjugated pneumococcal capsular saccharide; and a Tt-conjugated pneumococcal capsular saccharide.

The Dt/Tt-containing vaccine can, for instance, be any of the available commercial pediatric vaccines (e.g. PEDIACEL™, PENTACEL™, INFANRIX™, PEDIARIX™, DAPTACEL™, etc.), or a vaccine including immunogens from these vaccines. Thus the infant can receive one of: (a) a vaccine comprising Dt, Tt, pertussis toxoid, FHA, pertactin, pertussis fimbriae types 2 and 3, IPV, and Hib-Tt, with an aluminium phosphate adjuvant; (b) a vaccine comprising Dt, Tt, pertussis toxoid, FHA, and pertactin, with an aluminium hydroxide adjuvant; (c) a vaccine comprising Dt, Tt, pertussis toxoid, FHA, pertactin, HBsAg, and IPV, with aluminium hydroxide and aluminium phosphate adjuvants; or (d) a vaccine comprising Dt, Tt, pertussis toxoid, FHA, pertactin, and pertussis fimbriae types 2 and 3.

The vaccine should include an excess of Dt relative to Tt (as measured in Lf units). The excess is ideally at least 1.5-fold e.g. 2-fold or 2.5-fold, but the excess will not usually be more than 5-fold. A 2.5:1 ratio is useful e.g. 5 Lf of Dt for every 2 Lf of Tt.

The conjugated meningococcal/pneumococcal vaccine can be any of the available commercial vaccines which uses a CRM197 carrier e.g. MENVEO™, PREVNAR™, PREVNAR13™, etc. Thus the infant can receive (a) an unadjuvanted vaccine comprising CRM197-conjugated oligosaccharides from each of meningococcal serogroups A, C, W135 and Y; and/or one of (b1) a vaccine comprising CRM197-conjugated oligosaccharide from pneumococcal serotype 18C and CRM197-conjugated polysaccharides from each of pneumococcal serotypes 4, 6B, 9V, 14, 19F and 23F, with an aluminium phosphate adjuvant or (b2) a vaccine comprising CRM197-conjugated polysaccharides from each of pneumococcal serotypes 1, 3, 4, 5, 6A, 6B, 7F, 9V, 14, 18C, 19A, 19F and 23F, with an aluminium phosphate adjuvant.

Processes of Manufacture

The invention also provides processes for manufacturing the vaccines of the invention. These processes involve combining the relevant components (immunogens, adjuvants, carriers, etc.) in the desired ratios. In some embodiments the immunogens will be added individually, but in other embodiments the immunogens may already be in mixed form when they are used (e.g. a process might use a component which already includes mixed Dt and aP antigens). Similarly, in some embodiments the immunogens may be pre-adsorbed before being used in a process of the invention, but in other embodiments they may be added in unadsorbed form and can subsequently adsorb to adjuvant in the mixture.

Vaccines of the invention are made in bulk and are then sub-divided e.g. into unit doses.

A vaccine made by this process can be used as vaccine directly in a patient, or can be used as a component of a further combination vaccine.

Adjuvants

Vaccines of the invention will usually include an adjuvant. Adjuvants are included in current Dt- and Tt-containing vaccines, and in pneumococcal conjugate vaccines, and also in monovalent MenC conjugate vaccines, but are not included in current 4-valent MenACWY conjugate vaccines.

Where an adjuvant is included, this will usually comprise (i) at least one aluminium salt or (ii) an oil-in-water emulsion. Where a vaccine includes an aluminium salt adjuvant then preferably it does not also include an oil-in-water emulsion adjuvant. Conversely, where a vaccine includes an oil-in-water emulsion adjuvant then preferably it does not also include an aluminium salt adjuvant.

Where a vaccine includes aluminium salt adjuvant(s), between one and all of the immunogens in the vaccine can be adsorbed to the salt(s).

Aluminium Salt Adjuvants

Vaccines of the invention can include an aluminium salt adjuvant. Aluminium salt adjuvants currently in use are typically referred to either as “aluminium hydroxide” or as “aluminium phosphate” adjuvants. These are names of convenience, however, as neither is a precise description of the actual chemical compound which is present (e.g. see chapter 9 of reference 9, and chapter 4 of reference 10). The invention can use any of the “hydroxide” or “phosphate” salts that useful as adjuvants.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ (chapter 9 of ref. 9). The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants e.g. with needle-like particles with diameters about 2 nm. The PZC of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate. They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 0.99. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls (chapter 9 of ref. 9).

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate. Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The PZC of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

In solution both aluminium phosphate and hydroxide adjuvants tend to form stable porous aggregates 1-10 μm in diameter [11].

A vaccine can include a mixture of both an aluminium hydroxide and an aluminium phosphate, and components may be adsorbed to one or both of these salts.

An aluminium phosphate solution used to prepare a composition of the invention may contain a buffer (e.g. a phosphate or a histidine or a Tris buffer), but this is not always necessary. The aluminium phosphate solution is preferably sterile and pyrogen-free. The aluminium phosphate solution may include free aqueous phosphate ions e.g. present at a concentration between 1.0 and 20 mM, preferably between 5 and 15 mM, and more preferably about 10 mM. The aluminium phosphate solution may also comprise sodium chloride. The concentration of sodium chloride is preferably in the range of 0.1 to 100 mg/ml (e.g. 0.5-50 mg/ml, 1-20 mg/ml, 2-10 mg/ml) and is more preferably about 3±1 mg/ml. The presence of NaCl facilitates the correct measurement of pH prior to adsorption of antigens.

A composition of the invention ideally includes less than 0.85 mg Al⁺⁺⁺ per unit dose. In some embodiments of the invention a composition includes less than 0.5 mg Al⁺⁺⁺ per unit dose. The amount of Al⁺⁺⁺ can be lower than this e.g. <250 μg, <200 μg, <150 μg, <100 μg, <75 μg, <50 μg, <25 μg, <10 μg, etc.

Where a vaccine includes an aluminium-based adjuvant, settling of components may occur during storage. The composition should therefore be shaken prior to administration to a patient. The shaken composition will be a turbid white suspension.

Oil-in-Water Emulsion Adjuvants

In some embodiments a vaccine is adjuvanted with an oil-in-water emulsion. Various such emulsions are known e.g. MF59 and AS03 are both authorised in Europe.

Useful emulsion adjuvants typically include at least one oil and at least one surfactant, with the oil(s) and surfactant(s) being biodegradable (metabolisable) and biocompatible. The oil droplets in the emulsion generally have a sub-micron diameter, and these small sizes can readily be achieved with a microfluidiser to provide stable emulsions, or by alternative methods e.g. phase inversion. Emulsions in which at least 80% (by number) of droplets have a diameter of less than 220 nm are preferred, as they can be subjected to filter sterilization.

The emulsion can include oil(s) from an animal (such as fish) and/or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolisable and may therefore be used with the invention. The procedures for separation, purification, saponification and other means necessary for obtaining pure oils from animal sources are well known in the art.

Most fish contain metabolisable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred for use with the invention (see below). Squalane, the saturated analog to squalene, is also a useful oil. Fish oils, including squalene and squalane, are readily available from commercial sources or may be obtained by methods known in the art. Other preferred oils are the tocopherols (see below). Mixtures of oils can be used.

Preferred amounts of total oil (% by volume) in an adjuvant emulsion are between 1 and 20% e.g. between 2-10%. A squalene content of 5% by volume is particularly useful.

Surfactants can be classified by their ‘HLB’ (hydrophile/lipophile balance). Preferred surfactants of the invention have a HLB of at least 10 e.g. about 15. The invention can be used with surfactants including, but not limited to: the polyoxyethylene sorbitan esters surfactants (commonly referred to as the Tweens), especially polysorbate 20 or polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the Spans), such as sorbitan trioleate (Span 85) or sorbitan monolaurate.

Emulsions used with the invention preferably include non-ionic surfactant(s). Preferred surfactants for including in the emulsion are polysorbate 80 (polyoxyethylene sorbitan monooleate; Tween 80), Span 85 (sorbitan trioleate), lecithin or Triton X-100. Mixtures of surfactants can be used e.g. a mixture of polysorbate 80 and sorbitan trioleate. A combination of a polyoxyethylene sorbitan ester such as polysorbate 80 (Tween 80) and an octoxynol such as t-octylphenoxypolyethoxyethanol (Triton X-100) is also useful. Another useful combination comprises laureth 9 plus a polyoxyethylene sorbitan ester and/or an octoxynol. Where a mixture of surfactants is used then the HLB of the mixture is calculated according to their relative weightings (by volume) e.g. the preferred 1:1 mixture by volume of polysorbate 80 and sorbitan trioleate has a HLB of 8.4.

Preferred amounts of total surfactant (% by volume) in an adjuvant emulsion are between 0.1 and 2% e.g. between 0.25-2%. A total content of 1% by volume is particularly useful e.g. 0.5% by volume of polysorbate 80 and 0.5% by volume of sorbitan trioleate.

Useful emulsions can be prepared using known techniques e.g. see references 10 and 12-1318

Specific oil-in-water emulsion adjuvants useful with the invention include, but are not limited to:
A submicron emulsion of squalene, polysorbate 80, and sorbitan trioleate. The composition of the emulsion by volume can be about 5% squalene, about 0.5% polysorbate 80 and about 0.5% sorbitan trioleate. In weight terms, these ratios become 4.3% squalene, 0.5% polysorbate 80 and 0.48% sorbitan trioleate. This adjuvant is known as ‘MF59’ [19-21], as described in more detail in Chapter 10 of ref. 9 and chapter 12 of ref. 10. The MF59 emulsion advantageously includes citrate ions e.g. 10 mM sodium citrate buffer.
An emulsion of squalene, a tocopherol, and polysorbate 80. The emulsion may include phosphate buffered saline. These emulsions may have from 2 to 10% squalene, from 2 to 10% tocopherol and from 0.3 to 3% polysorbate 80, and the weight ratio of squalene:tocopherol is preferably ≦1 (e.g. 0.90) as this can provide a more stable emulsion. Squalene and polysorbate 80 may be present volume ratio of about 5:2, or at a weight ratio of about 11:5. Thus the three components (squalene, tocopherol, polysorbate 80) may be present at a weight ratio of 1068:1186:485 or around 55:61:25. This adjuvant is known as ‘AS03’. Another useful emulsion of this type may comprise, per human dose, 0.5-10 mg squalene, 0.5-11 mg tocopherol, and 0.1-4 mg polysorbate 80 [22] e.g. in the ratios discussed above.
An emulsion in which a saponin (e.g. QuilA or QS21) and a sterol (e.g. a cholesterol) are associated as helical micelles [23].
An emulsion having from 0.5-50% of an oil, 0.1-10% of a phospholipid, and 0.05-5% of a non-ionic surfactant. As described in reference 24, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [25]. The emulsion may also include one or more of: alditol; a cryoprotective agent (e.g. a sugar, such as dodecylmaltoside and/or sucrose); and/or an alkylpolyglycoside. It may also include a TLR4 agonist, such as one whose chemical structure does not include a sugar ring [26]. Such emulsions may be lyophilized. The ‘AF03’ product is one such emulsion.

Preferred oil-in-water emulsions used with the invention comprise squalene and polysorbate 80.

The emulsions may be mixed with antigens during vaccine manufacture, or they may be mixed extemporaneously at the time of delivery. Thus, in some embodiments, the adjuvant and antigens may be kept separately in a packaged or distributed vaccine, ready for final formulation at the time of use. At the time of mixing (whether during bulk manufacture, or at the point of use) the antigen will generally be in an aqueous form, such that the final vaccine is prepared by mixing two liquids. The volume ratio of the two liquids for mixing can vary (e.g. between 5:1 and 1:5) but is generally about 1:1. If emulsion and antigen are stored separately in a kit then the product may be presented as a vial containing emulsion and a vial containing aqueous antigen, for mixing to give adjuvanted liquid vaccine (monodose or multi-dose).

Preferred emulsions of the invention include squalene oil. This is usually prepared from shark oil but alternative sources are known e.g. see references 27 (yeast) and 28 (olive oil). Squalene which contains less than 661 picograms of PCBs per gram of squalene (TEQ) is preferred for use with the invention, as disclosed in reference 29. The emulsions are preferably made from squalene of high purity e.g. prepared by double-distillation as disclosed in reference 30.

Where a composition includes a tocopherol, any of the α, β, γ, δ, ε or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols have antioxidant properties that may help to stabilize the emulsions [31]. A preferred α-tocopherol is DL-α-tocopherol, and a preferred salt of this tocopherol is the succinate.

Vaccine Compositions

In addition to the antigen and adjuvant components discussed above, vaccines of the invention may comprise further non-antigenic component(s). These can include carriers, excipients, buffers, etc. These non-antigenic components may have various sources. For example, they may be present in one of the antigen or adjuvant materials that is used during manufacture or may be added separately from those components.

Preferred vaccines of the invention include one or more pharmaceutical carrier(s) and/or excipient(s).

To control tonicity, it is preferred to include a physiological salt, such as a sodium salt. Sodium chloride (NaCl) is preferred, which may be present at between 1 and 20 mg/ml.

Vaccines will generally have an osmolality of between 200 mOsm/kg and 400 mOsm/kg, preferably between 240-360 mOsm/kg, and will more preferably fall within the range of 280-320 mOsm/kg. Osmolality has previously been reported not to have an impact on pain caused by vaccination [32], but keeping osmolality in this range is nevertheless preferred.

Vaccines of the invention may include one or more buffers. Typical buffers include: a phosphate buffer; a Tris buffer; a borate buffer; a succinate buffer; a histidine buffer; or a citrate buffer. Buffers will typically be included in the 5-20 mM range.

The pH of a vaccine of the invention will generally be between 6.0 and 7.5. A manufacturing process may therefore include a step of adjusting the pH of a composition prior to packaging. Aqueous compositions administered to a patient can have a pH of between 5.0 and 7.5, and more typically between 5.0 and 6.0 for optimum stability; where a diphtheria toxoid and/or tetanus toxoid is present, the pH is ideally between 6.0 and 7.0.

Vaccines of the Invention are Preferably Sterile.

Vaccines of the invention are preferably non-pyrogenic e.g. containing <1 EU (endotoxin unit, a standard measure; 1 EU is equal to 0.2 ng FDA reference standard Endotoxin EC-2 ‘RSE’) per dose, and preferably <0.1 EU per dose.

Vaccines of the Invention are Preferably Gluten Free.

If a vaccine includes adsorbed component then it may be a suspension with a cloudy appearance. This appearance means that microbial contamination is not readily visible, and so the vaccine preferably contains an antimicrobial agent. This is particularly important when the vaccine is packaged in multidose containers. Preferred antimicrobials for inclusion are 2-phenoxyethanol and thimerosal. It is preferred, however, not to use mercurial preservatives (e.g. thimerosal) during a process of the invention. Thus, between 1 and all of the components mixed in a process may be substantially free from mercurial preservative. However, the presence of trace amounts may be unavoidable if a component was treated with such a preservative before being used in the invention.

For safety, however, it is preferred that the final composition contains less than about 25 ng/ml mercury. More preferably, the final vaccine product contains no detectable thimerosal. This will generally be achieved by removing the mercurial preservative from an antigen preparation prior to its addition in the process of the invention or by avoiding the use of thimerosal during the preparation of the components used to make the composition. Mercury-free vaccines are preferred.

Vaccines of the Invention Will Usually be in Aqueous Form.

During manufacture, dilution of components to give desired final concentrations will usually be performed with WFI (water for injection), or with buffer.

The invention can provide bulk material which is suitable for packaging into individual doses, which can then be distributed for administration to patients. Concentrations discussed above are typically concentrations in final packaged dose, and so concentrations in bulk vaccine may be higher (e.g. to be reduced to final concentrations by dilution).

Vaccines of the invention are administered to patients in unit doses i.e. the amount of a vaccine given to a single patient in a single administration (e.g. a single injection is a unit dose). Where a vaccine is administered as a liquid then a unit dose typically has a volume of 0.5 ml. This volume will be understood to include normal variance e.g. 0.5 ml±0.05 ml. For multidose situations, multiple dose amounts will be extracted and packaged together in a single container e.g. 5 ml for a 10-dose multidose container (or 5.5 ml with 10% overfill).

Residual material from individual antigenic components may also be present in trace amounts in the final vaccine. For example, if formaldehyde is used to prepare the toxoids of diphtheria, tetanus and pertussis then the final vaccine product may retain trace amounts of formaldehyde (e.g. less than 10 μg/ml, preferably <5 μg/ml). Media or stabilizers may have been used during poliovirus preparation (e.g. Medium 199), and these may carry through to the final vaccine. Similarly, free amino acids (e.g. alanine, arginine, aspartate, cysteine and/or cystine, glutamate, glutamine, glycine, histidine, proline and/or hydroxyproline, isoleucine, leucine, lysine, methionine, phenylalanine, serine, threonine, tryptophan, tyrosine and/or valine), vitamins (e.g. choline, ascorbate, etc.), disodium phosphate, monopotassium phosphate, calcium, glucose, adenine sulfate, phenol red, sodium acetate, potassium chloride, etc. may be retained in the final vaccine at ≦100 μg/ml, preferably <10 μg/ml, each. Other components from antigen preparations, such as neomycin (e.g. neomycin sulfate, particularly from a poliovirus component), polymyxin B (e.g. polymyxin B sulfate, particularly from a poliovirus component), etc. may also be present at sub-nanogram amounts per dose. A further possible component of the final vaccine which originates in the antigen preparations arises from less-than-total purification of antigens. Small amounts of B. pertussis, C. diphtheriae, C. tetani and S. cerevisiae proteins and/or genomic DNA may therefore be present. To minimize the amounts of these residual components, antigen preparations are preferably treated to remove them prior to the antigens being used with the invention.

Where a poliovirus component is used, it will generally have been grown on Vero cells. The final vaccine preferably contains less than 10 ng/ml, preferably <1 ng/ml e.g. <500 μg/ml or <50 μg/ml of Vero cell DNA e.g. less than 10 ng/ml of Vero cell DNA that is >50 base pairs long.

Vaccines of the invention are presented for use in containers. Suitable containers include vials and disposable syringes (preferably sterile ones). Processes of the invention may comprise a step of packaging the vaccine into containers for use. Suitable containers include vials and disposable syringes (preferably sterile ones).

The invention also provides a delivery device (e.g. syringe, nebuliser, sprayer, inhaler, dermal patch, etc.) containing a vaccine of the invention e.g. containing a unit dose. This device can be used to administer the vaccine to an infant.

The invention also provides a sterile container (e.g. a vial) containing a vaccine of the invention e.g. containing a unit dose.

The invention also provides a unit dose of a vaccine of the invention.

The invention also provides a hermetically sealed container containing a vaccine of the invention. Suitable containers include e.g. a vial.

Where a vaccine of the invention is presented in a vial, this is preferably made of a glass or plastic material. The vial is preferably sterilized before the composition is added to it. To avoid problems with latex-sensitive patients, vials may be sealed with a latex-free stopper. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. Preferred vials are made of colorless glass.

A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed.

Where the vaccine is packaged into a syringe, the syringe will not normally have a needle attached to it, although a separate needle may be supplied with the syringe for assembly and use. Safety needles are preferred. 1-inch 23-gauge, 1-inch 25-gauge and ⅝-inch 25-gauge needles are typical. Syringes may be provided with peel-off labels on which the lot number and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of butyl rubber. If the syringe and needle are packaged separately then the needle is preferably fitted with a butyl rubber shield. Grey butyl rubber is preferred. Preferred syringes are those marketed under the trade name “Tip-Lok”™.

Where a glass container (e.g. a syringe or a vial) is used, then it is preferred to use a container made from a borosilicate glass rather than from a soda lime glass.

After a vaccine is packaged into a container, the container can then be enclosed within a box for distribution e.g. inside a cardboard box, and the box will be labeled with details of the vaccine e.g. its trade name, a list of the antigens in the vaccine (e.g. ‘hepatitis B recombinant’, etc.), the presentation container (e.g. ‘Disposable Prefilled Tip-Lok Syringes’ or ‘10×0.5 ml Single-Dose Vials’), its dose (e.g. ‘each containing one 0.5 ml dose’), warnings (e.g. ‘For Adult Use Only’ or ‘For Pediatric Use Only’), an expiration date, an indication, a patent number, etc. Each box might contain more than one packaged vaccine e.g. five or ten packaged vaccines (particularly for vials).

The vaccine may be packaged together (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

The packaged vaccine is preferably stored at between 2° C. and 8° C. It should not be frozen.

Where a component is lyophilised it generally includes non-active components which were added prior to freeze-drying e.g. as stabilizers. Preferred stabilizers for inclusion are lactose, sucrose and mannitol, as well as mixtures thereof e.g. lactose/sucrose mixtures, sucrose/mannitol mixtures, etc. A final vaccine obtained by aqueous reconstitution of the lyophilised material may thus contain lactose and/or sucrose. It is preferred to use amorphous excipients and/or amorphous buffers when preparing lyophilised vaccines [33].

Methods of Treatment, and Administration of the Vaccine

Vaccines of the invention are suitable for administration to human infants, and the invention provides a method of raising an immune response in an infant, comprising the step of administering a composition of the invention to the patient.

The invention also provides a vaccine of the invention for use in medicine. The composition may be administered as variously described herein. Thus the vaccines are provided for use in any of the immunisation methods disclosed herein e.g. for use in methods for immunising infants against multiple pathogens.

The invention also provides the use of the antigens mentioned herein (and, optionally, an adjuvant) in the manufacture of a medicament for raising an immune response in an infant. The medicament is ideally a composition as variously described elsewhere herein, and it can be administered as variously described herein. The antigens which are used in manufacture determine the effect of the immune response which is raised by the infant.

The vaccines of the invention are used for active immunisation. The immune responses raised by these methods, uses and compositions are ideally protective, and vaccines of the invention can be used in the prevention of various diseases. When a vaccine includes a diphtheria toxoid (whether conjugated or unconjugated) it can protect against diphtheria. When a vaccine includes a tetanus toxoid (whether conjugated or unconjugated) it can protect against tetanus. When a vaccine includes acellular pertussis antigen(s) it can protect against pertussis (whooping cough). When a vaccine includes HBsAg it can protect against hepatitis B. When a vaccine includes IPV it can protect against poliomyelitis. When a vaccine includes a Hib capsular saccharide it can protect against disease caused by Haemophilus influenzae type b. When a vaccine includes a meningococcal capsular saccharide from a particular serogroup(s) it can protect against meningococcal diseases (in particular, invasive meningococcal diseases) caused by Neisseria meningitidis of that serogroup(s). When a vaccine includes a pneumococcal capsular saccharide from a particular serotype(s) it can protect against diseases (in particular, invasive diseases) caused by Streptococcus pneumoniae of that serotype(s), and it may also protect against otitis media caused by those serotype(s).

Vaccines of the invention are useful for primary immunisation of infants. In order to have full efficacy, a typical primary immunisation schedule (particularly for an infant) may involve administering more than one dose. For example, doses may be at: 0 & 6 months (time 0 being the first dose); at 0, 1, 2 & 6 months; at day 0, day 21 and then a third dose between 6 & 12 months; at 2, 4 & 6 months; at 3, 4 & 5 months; at 6, 10 & 14 weeks; at 2, 3 & 4 months; or at 0, 1, 2, 6 & 12 months.

Vaccines of the invention can also be used later in life as booster doses e.g. for children in the second year of life, for an adolescent, or for an adult.

Vaccines of the invention can be administered by intramuscular injection e.g. into the arm or leg. Injection into the anterolateral aspect of the thigh or the deltoid muscle of the upper arm is typical.

Diphtheria Toxoid

Diphtheria is caused by Corynebacterium diphtheriae, a Gram-positive non-sporing aerobic bacterium. This organism expresses a prophage-encoded ADP-ribosylating exotoxin (‘diphtheria toxin’), which can be treated (e.g. using formaldehyde) to give a toxoid that is no longer toxic but that remains antigenic and is able to stimulate the production of specific anti-toxin antibodies after injection. Diphtheria toxoids are disclosed in more detail in chapter 13 of reference 4. Preferred diphtheria toxoids are those prepared by formaldehyde treatment. The diphtheria toxoid can be obtained by growing C. diphtheriae in growth medium (e.g. Fenton medium, or Linggoud & Fenton medium), which may be supplemented with bovine extract, followed by formaldehyde treatment, ultrafiltration and precipitation. The toxoided material may then be treated by a process comprising sterile filtration and/or dialysis.

A composition should include enough diphtheria toxoid to elicit circulating diphtheria antitoxin levels of at least 0.01 IU/ml. Quantities of diphtheria toxoid are generally measured in the ‘Lf’ unit (“flocculating units”, or the “limes flocculating dose”, or the “limit of flocculation”), defined as the amount of toxin/toxoid which, when mixed with one International Unit of antitoxin, produces an optimally flocculating mixture [34,35]. For example, the NIBSC supplies ‘Diphtheria Toxoid, Plain’ [36], which contains 300 LF per ampoule, and also supplies ‘The 1st International Reference Reagent For Diphtheria Toxoid For Flocculation Test’ [37] which contains 900 Lf per ampoule. The concentration of diphtheria toxoid in a composition can readily be determined using a flocculation assay by comparison with a reference material calibrated against such reference reagents.

The immunizing potency of diphtheria toxoid in a composition is generally expressed in international units (IU). The potency can be assessed by comparing the protection afforded by a composition in laboratory animals (typically guinea pigs) with a reference vaccine that has been calibrated in IUs.

NIBSC supplies the ‘Diphtheria Toxoid Adsorbed Third International Standard 1999’ [38,39], which contains 160 IU per ampoule, and is suitable for calibrating such assays.

The conversion between IU and Lf systems depends on the particular toxoid preparation.

Vaccines of the invention typically include, per unit dose, between 10-35 Lf diphtheria toxoid per unit dose e.g. between 15-30 Lf, such as 15, 25 or 30 Lf. By IU measurements, vaccines of the invention will generally include >25 IU diphtheria toxoid per unit dose.

Where a vaccine includes diphtheria toxoid, it should include enough to meet the European Pharmacopoeia requirements for diphtheria vaccination (protection of guinea pigs against lethal challenge by diphtheria toxin). Where the diphtheria toxoid is a carrier protein in a saccharide conjugate, the ratio of saccharide:toxoid in the conjugate will vary such that the conjugate can provide enough diphtheria toxoid to meet the minimum potency requirement for diphtheria protection, and enough saccharide to provide the required dose (e.g. between 5-15 μg of Hib saccharide per dose).

If a composition includes an aluminium salt adjuvant then diphtheria toxoid in the composition is preferably adsorbed (more preferably totally adsorbed) onto it, and preferably onto an aluminium hydroxide adjuvant.

Tetanus Toxoid

Tetanus is caused by Clostridium tetani, a Gram-positive, spore-forming bacillus. This organism expresses an endopeptidase (‘tetanus toxin’), which can be treated to give a toxoid that is no longer toxic but that remains antigenic and is able to stimulate the production of specific anti-toxin antibodies after injection. Tetanus toxoids are disclosed in more detail in chapter 27 of reference 4. Preferred tetanus toxoids are those prepared by formaldehyde treatment. The tetanus toxoid can be obtained by growing C. tetani in growth medium (e.g. a Latham medium derived from bovine casein), followed by formaldehyde treatment, ultrafiltration and precipitation. The material may then be treated by a process comprising sterile filtration and/or dialysis.

A composition should include enough tetanus toxoid to elicit circulating tetanus antitoxin levels of at least 0.01 IU/ml. Quantities of tetanus toxoid are generally expressed in ‘Lf’ units (see above), defined as the amount of toxoid which, when mixed with one International Unit of antitoxin, produces an optimally flocculating mixture [34]. The NIBSC supplies ‘The 1st International Reference Reagent for Tetanus Toxoid For Flocculation Test’ [40] which contains 1000 LF per ampoule, by which measurements can be calibrated.

The immunizing potency of tetanus toxoid is measured in international units (IU), assessed by comparing the protection afforded by a composition in laboratory animals (typically guinea pigs) with a reference vaccine e.g. using NIBSC's ‘Tetanus Toxoid Adsorbed Third International Standard 2000’ [41,42], which contains 469 IU per ampoule.

The conversion between IU and Lf systems depends on the particular toxoid preparation.

Vaccines of the invention typically include between 4-15 Lf tetanus toxoid per unit dose e.g. between 5-10 Lf, such as 5 or 10 Lf. By IU measurements, vaccines of the invention will generally include >40 IU tetanus toxoid per unit dose.

Where a vaccine includes tetanus toxoid, it should include enough to meet the European Pharmacopoeia requirements for tetanus vaccination (protection of mice against lethal challenge by tetanus toxin). Where the tetanus toxoid is a carrier protein in a saccharide conjugate, the ratio of saccharide:toxoid in the conjugate will vary such that the conjugate can provide enough tetanus toxoid to meet the minimum potency requirement for tetanus protection, and enough saccharide to provide the required dose (e.g. between 5-15 μg of Hib saccharide per dose).

If a composition includes an aluminium salt adjuvant then tetanus toxoid in the composition is preferably adsorbed (sometimes totally adsorbed) onto an aluminium salt, preferably onto an aluminium hydroxide adjuvant.

Acellular Pertussis Antigens

Bordetella pertussis causes whooping cough. Compositions of the invention include an acellular (“aP”) pertussis antigen i.e. a defined mixture of purified pertussis antigens, rather than a cellular lysate. The vaccine will typically include at least two of pertussis toxoid (′PT′ i.e. a detoxified form of pertussis toxin), filamentous hemagglutinin (FHA), and/or pertactin (also known as the ‘69 kiloDalton outer membrane protein’). It can also optionally include fimbriae types 2 and 3. Preparation of these various aP antigens is well known in the art.

PT can be detoxified by treatment with formaldehyde and/or glutaraldehyde, and FHA and pertactin can also be treated in the same way. As an alternative to chemical detoxification of PT, the invention can use a mutant PT in which wild-type enzymatic activity has been reduced by mutagenesis [43] e.g. the 9K/129G double mutant [44].

Quantities of acellular pertussis antigens are usually expressed in micrograms. Vaccines of the invention typically include between 5-30 μg PT per unit dose (e.g. 5, 7.5, 20 or 25 μg), between 2.5-25 μg FHA per unit dose (e.g. 2.5, 5, 10, 20 or 25 μg), and between 2.5-10 μg pertactin per unit dose (e.g. 2.5, 3, 8 or 10 μg), A composition normally contains <80 μg per unit dose of total acellular pertussis antigens. Each individual antigen will usually be present at <30 μg per unit dose.

It is usual that each of PT, FHA and pertactin are present in a composition of the invention. These may be present at various ratios (by mass), such as PT:FHA:p69 ratios of 20:20:3 or 25:25:8. It is usual to have a mass excess of FHA relative to pertactin if both are present.

If a composition includes an aluminium salt adjuvant then PT in the composition is preferably adsorbed (sometimes totally adsorbed) onto an aluminium salt, preferably onto an aluminium hydroxide adjuvant. Any FHA can also be adsorbed to the aluminium salt. Any pertactin can be adsorbed to the aluminium salt adjuvant, but the presence of pertactin normally means that the composition requires the presence of aluminium hydroxide to ensure stable adsorption [45].

Inactivated Poliovirus Antigen (IPV)

Poliomyelitis can be caused by one of three types of poliovirus. The three types are similar and cause identical symptoms, but they are antigenically very different and infection by one type does not protect against infection by others. As explained in chapter 24 of reference 4, it is therefore preferred to use three poliovirus antigens with the invention—poliovirus Type 1 (e.g. Mahoney strain), poliovirus Type 2 (e.g. MEF-1 strain), and poliovirus Type 3 (e.g. Saukett strain). As an alternative to these strains (“Salk” strains), Sabin strains of types 1 to 3 can be used e.g. as discussed in references 46 & 47. These strains can be more potent than the normal Salk strains.

Polioviruses may be grown in cell culture. A preferred culture uses a Vero cell line, which is a continuous cell line derived from monkey kidney. Vero cells can conveniently be cultured microcarriers. Culture of the Vero cells before and during viral infection may involve the use of bovine-derived material, such as calf serum, and of lactalbumin hydrolysate (e.g. obtained by enzymatic degradation of lactalbumin). Such bovine-derived material should be obtained from sources which are free from BSE or other TSEs.

After growth, virions may be purified using techniques such as ultrafiltration, diafiltration, and chromatography. Prior to administration to patients, polioviruses must be inactivated, and this can be achieved by treatment with formaldehyde before the viruses are used in the process of the invention.

The viruses are preferably grown, purified and inactivated individually, and are then combined to give a bulk mixture for use with the invention.

Quantities of IPV are typically expressed in the ‘DU’ unit (the “D-antigen unit” [48]). Where all three of Types 1, 2 and 3 poliovirus are present the three antigens can be present at a DU ratio of 5:1:4 respectively, or at any other suitable ratio e.g. a ratio of 15:32:45 when using Sabin strains [46]. Typical amounts of Salk IPV strains per unit dose are 40 DU type 1, 8 DU type 2 and 32 DU type 3, although lower doses can also be used. A low amount of antigen from Sabin strains is particularly useful, with ≦15 DU type 1, ≦5 DU type 2, and ≦25 DU type 3 (per unit dose).

If a composition includes an aluminium salt adjuvant then IPV antigens are often not pre-adsorbed to any adjuvant before they are used in a process of the invention, but after formulation they may become adsorbed onto the aluminium salt(s).

Hepatitis B Virus Surface Antigen

Hepatitis B virus (HBV) is one of the known agents which causes viral hepatitis. The HBV virion consists of an inner core surrounded by an outer protein coat or capsid, and the viral core contains the viral DNA genome. The major component of the capsid is a protein known as HBV surface antigen or, more commonly, ‘HBsAg’, which is typically a 226-amino acid polypeptide with a molecular weight of ˜24 kDa. All existing hepatitis B vaccines contain HBsAg, and when this antigen is administered to a normal vaccinee it stimulates the production of anti-HBsAg antibodies which protect against HBV infection.

For vaccine manufacture, HBsAg can be made in two ways. The first method involves purifying the antigen in particulate form from the plasma of chronic hepatitis B carriers, as large quantities of HBsAg are synthesized in the liver and released into the blood stream during an HBV infection. The second way involves expressing the protein by recombinant DNA methods. HBsAg for use with the method of the invention is recombinantly expressed e.g. in yeast or CHO cells. Suitable yeasts include Saccharomyces (such as S. cerevisiae) or Hanensula (such as H. polymorpha) hosts.

Unlike native HBsAg (i.e. as in the plasma-purified product), yeast-expressed HBsAg is generally non-glycosylated, and this is the most preferred form of HBsAg for use with the invention. Yeast-expressed HBsAg is highly immunogenic and can be prepared without the risk of blood product contamination.

The HBsAg will generally be in the form of substantially-spherical particles (average diameter of about 20 nm), including a lipid matrix comprising phospholipids. Yeast-expressed HBsAg particles may include phosphatidylinositol, which is not found in natural HBV virions. The particles may also include a non-toxic amount of LPS in order to stimulate the immune system [49]. The particles may retain non-ionic surfactant (e.g. polysorbate 20) if this was used during disruption of yeast [50].

A preferred method for HBsAg purification involves, after cell disruption: ultrafiltration; size exclusion chromatography; anion exchange chromatography; ultracentrifugation; desalting; and sterile filtration. Lysates may be precipitated after cell disruption (e.g. using a polyethylene glycol), leaving HBsAg in solution, ready for ultrafiltration.

After purification HBsAg may be subjected to dialysis (e.g. with cysteine), which can be used to remove any mercurial preservatives such as thimerosal that may have been used during HBsAg preparation [51]. Thimerosal-free preparation is preferred.

The HBsAg is preferably from HBV subtype adw2.

Quantities of HBsAg are typically expressed in micrograms. If a vaccine of the invention includes HBsAg then a normal quantity per unit dose is between 5-25 μg e.g. 10 μg or 20 μg.

If a composition includes an aluminium salt adjuvant then HBsAg can be adsorbed onto it (preferably adsorbed onto an aluminium phosphate adjuvant).

Hib Conjugates

Haemophilus influenzae type b (‘Hib’) causes bacterial meningitis. Hib vaccines are typically based on the ‘PRP’ capsular saccharide antigen (e.g. chapter 14 of ref. 4), the preparation of which is well documented (e.g. references 52 to 61). The Hib saccharide is conjugated to a carrier protein in order to enhance its immunogenicity, especially in children. Typical carrier proteins are tetanus toxoid, diphtheria toxoid, the CRM197 derivative of diphtheria toxoid, or the outer membrane protein complex from serogroup B meningococcus. Tetanus toxoid is a useful carrier, as used in the product commonly referred to as ‘PRP-T’ or ‘Hib-T’ i.e. purified Hib polyribosylribitol phosphate capsular polysaccharide covalently bound to tetanus protein. PRP-T can be made by activating a Hib capsular polysaccharide using cyanogen bromide, coupling the activated saccharide to an adipic acid linker (such as (1-ethyl-3-(3-dimethylaminopropyl) carbodiimide), typically the hydrochloride salt), and then reacting the linker-saccharide entity with a tetanus toxoid carrier protein. CRM197 is another useful carrier for Hib conjugate in compositions of the invention (e.g. as seen in the ‘HbOC’ and ‘Vaxem-Hib’ products).

The saccharide moiety of the conjugate may comprise full-length polyribosylribitol phosphate (PRP) as prepared from Hib bacteria, and/or fragments of full-length PRP. Conjugates with a saccharide:protein ratio (w/w) of between 1:5 (i.e. excess protein) and 5:1 (i.e. excess saccharide) may be used e.g. ratios between 1:2 and 5:1 and ratios between 1:1.25 and 1:2.5. In preferred vaccines, however, the weight ratio of saccharide to carrier protein is between 1:2.5 and 1:3.5. In vaccines where tetanus toxoid is present both as an antigen and as a carrier protein then the weight ratio of saccharide to carrier protein in the conjugate may be between 1:0.3 and 1:2 [62]. Administration of the Hib conjugate preferably results in an anti-PRP antibody concentration of ≧0.15 μg/ml, and more preferably ≧1 μg/ml, and these are the standard response thresholds.

Quantities of Hib antigens are typically expressed in micrograms of saccharide. If a composition of the invention includes a Hib antigen then a normal quantity per unit dose is between 5-15 μg e.g. 10 μg or 12 μg.

As mentioned above, the ratio of capsular saccharide to carrier protein in a conjugate can vary, such that the conjugate can provide enough toxoid to meet the minimum potency requirement for protection, and enough saccharide to provide the required dose. This ratio will vary according to the toxoid's specific potency. Thus the saccharide:toxoid mass ratio in a Hib conjugate could vary, from having excess saccharide (by mass), equal amounts of both (e.g. 10 μg Hib saccharide conjugated to 10 μg of toxoid), or excess carrier (by mass). Excess carrier protein is typical.

If a vaccine includes an aluminium salt adjuvant then Hib antigen can be adsorbed onto it or can be unadsorbed.

Meningococcal Capsular Saccharide Conjugate(s)

Where a composition includes a Neisseria meningitidis capsular saccharide conjugate there may be one or more than one such conjugate. Including 2, 3, or 4 of serogroups A, C, W135 and Y is typical e.g. A+C, A+W135, A+Y, C+W135, C+Y, W135+Y, A+C+W135, A+C+Y, A+W135+Y, A+C+W135+Y, etc. Components including saccharides from all four of serogroups A, C, W135 and

Y are useful, as in the MENVEO™, MENACTRA™ and NIMENRIX™ products. It is also possible to include a conjugate of a serogroup X N. meningitidis capsular saccharide.

Where conjugates from more than one serogroup are included, these are preferably prepared separately, conjugated separately, and then combined. They may be present at substantially equal masses e.g. the mass of each serogroup's saccharide is within +10% of each other. A typical quantity per serogroup is between 1 μg and 20 μg e.g. between 2 and 10 μg per serogroup, or about 4 μg or about 5 μg or about 10 μg. As an alternative to a substantially equal ratio, a double mass of serogroup A saccharide may be used (as in the MENVEO™ product).

Administration of a conjugate preferably results in an increase in serum bactericidal assay (SBA) titre for the relevant serogroup of at least 4-fold, and preferably at least 8-fold. SBA titres can be measured using baby rabbit complement or human complement [63].

The capsular saccharide of serogroup A meningococcus (‘MenA’) is a homopolymer of (α1Θα6)-linked N-acetyl-D-mannosamine-1-phosphate, with partial O-acetylation in the C3 and C4 positions. Acetylation at the C-3 position can be 70-95%. Conditions used to purify the saccharide can result in de-O-acetylation (e.g. under basic conditions), but it is useful to retain OAc at this C-3 position. In some embodiments, at least 50% (e.g. at least 60%, 70%, 80%, 90%, 95% or more) of the mannosamine residues in a serogroup A saccharides are O-acetylated at the C-3 position. Acetyl groups can be replaced with blocking groups to prevent hydrolysis [64], and such modified saccharides are still serogroup A saccharides within the meaning of the invention.

The serogroup C (‘MenC’) capsular saccharide is a homopolymer of (a 2→9)-linked sialic acid (N-acetyl neuraminic acid, or ‘NeuNAc’). The saccharide structure is written as →9)-Neup NAc 7/8 OAc-(α2→. Most serogroup C strains have O-acetyl groups at C-7 and/or C-8 of the sialic acid residues, but about 15% of clinical isolates lack these O-acetyl groups [65,66]. The presence or absence of OAc groups generates unique epitopes, and the specificity of antibody binding to the saccharide may affect its bactericidal activity against O-acetylated (OAc−) and de-O-acetylated (OAc+) strains [67-69]. Serogroup C saccharides used with the invention may be prepared from either OAc+ or OAc− strains. Licensed MenC conjugate vaccines include both OAc− (NEISVAC-C™) and OAc+ (MENJUGATE™ & MENINGITEC™) saccharides. In some embodiments, strains for production of serogroup C conjugates are OAc+ strains, e.g. of serotype 16, serosubtype P1.7a,1, etc. Thus C:16:P1.7a,1 OAc+ strains may be used. OAc+ strains in serosubtype P1.1 are also useful, such as the C11 strain. Preferred MenC saccharides are taken from OAc+ strains, such as strain C11.

The serogroup W135 (‘MenW’) capsular saccharide is a polymer of sialic acid-galactose disaccharide units. Like the serogroup C saccharide, it has variable O-acetylation, but at sialic acid 7 and 9 positions [70]. The structure is written: →4)-D-Neup5Ac(7/9OAc)-α-(2→6)-D-Gal-α-(1→.

The serogroup X (‘MenX’) capsular saccharide is a polymer of α1→4-linked N-acetylglucosamine 1-phosphate. The serogroup X structure is written as: →4)-α-D-GlcpNAc-(1→OPO₃→.

The serogroup Y (‘MenY’) saccharide is similar to the serogroup W135 saccharide, except that the disaccharide repeating unit includes glucose instead of galactose. Like serogroup W135, it has variable O-acetylation at sialic acid 7 and 9 positions [70]. The serogroup Y structure is written as: →4)-D-Neup5Ac(7/9OAc)-α-(2→6)-D-Glc-α-(1→.

The saccharides used according to the invention may be O-acetylated as described above (e.g. with the same O-acetylation pattern as seen in native capsular saccharides), or they may be partially or totally de-O-acetylated at one or more positions of the saccharide rings, or they may be hyper-O-acetylated relative to the native capsular saccharides. For example, reference 71 reports the use of serogroup Y saccharides that are more than 80% de-O-acetylated.

The saccharide moieties in meningococcal conjugates may comprise full-length saccharides as prepared from meningococci, and/or may comprise fragments of full-length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. One depolymerisation method involves the use of hydrogen peroxide [72]. Hydrogen peroxide is added to a saccharide (e.g. to give a final H₂O₂ concentration of 1%), and the mixture is then incubated (e.g. at about 55° C.) until a desired chain length reduction has been achieved. Another depolymerisation method involves acid hydrolysis [73], and other methods include microfluidisation or sonication [74]. Other depolymerisation methods are known in the art. The saccharides used to prepare conjugates for use according to the invention may be obtainable by any of these depolymerisation methods. Depolymerisation can be used in order to provide an optimum chain length for immunogenicity and/or to reduce chain length for physical manageability of the saccharides. In some embodiments, saccharides have the following range of average degrees of polymerisation (Dp): A=10-20; C=12-22; W135=15-25; Y=15-25. In terms of molecular weight, rather than Dp, useful ranges are, for all serogroups: <100 kDa; 5 kDa-75 kDa; 7 kDa-50 kDa; 8 kDa-35 kDa; 12 kDa-25 kDa; 15 kDa-22 kDa. In other embodiments, the average molecular weight for saccharides from each of meningococcal serogroups A, C, W135 and Y may be more than 50 kDa e.g. ≧75 kDa, ≧100 kDa, ≧110 kDa, ≧120 kDa, ≧130 kDa, etc. [74], and even up to 1500 kDa, in particular as determined by MALLS. For instance: a MenA saccharide may be in the range 50-500 kDa e.g. 60-80 kDa; a MenC saccharide may be in the range 100-210 kDa; a MenW135 saccharide may be in the range 60-190 kDa e.g. 120-140 kDa; and/or a MenY saccharide may be in the range 60-190 kDa e.g. 150-160 kDa.

If a component or composition includes both Hib and meningococcal conjugates then, in some embodiments, the mass of Hib saccharide can be substantially the same as the mass of a particular meningococcal serogroup saccharide. In some embodiments, the mass of Hib saccharide will be more than (e.g. at least 1.5×) the mass of a particular meningococcal serogroup saccharide. In some embodiments, the mass of Hib saccharide will be less than (e.g. at least 1.5× less) the mass of a particular meningococcal serogroup saccharide.

Where a composition includes saccharide from more than one meningococcal serogroup, there is an mean saccharide mass per serogroup. If substantially equal masses of each serogroup are used then the mean mass will be the same as each individual mass; where non-equal masses are used then the mean will differ e.g. with a 10:5:5:5 μg amount for a MenACWY mixture, the mean mass is 6.25 μg per serogroup. In some embodiments, the mass of Hib saccharide will be substantially the same as the mean mass of meningococcal saccharide per serogroup. In some embodiments, the mass of Hib saccharide will be more than (e.g. at least 1.5×) the mean mass of meningococcal saccharide per serogroup. In some embodiments, the mass of Hib saccharide will be less than (e.g. at least 1.5×) the mean mass of meningococcal saccharide per serogroup [75].

As mentioned above, the ratio of capsular saccharide to carrier protein in a conjugate can vary, such that the conjugate can provide enough toxoid to meet the minimum potency requirement for protection, and enough saccharide to provide the required dose. This ratio will vary according to the toxoid's specific potency. Thus the saccharide:toxoid mass ratio in a meningococcal conjugate could vary, from having excess saccharide (by mass), equal amounts of both (e.g. 10 μg meningococcal saccharide conjugated to 10 μg of toxoid), or excess carrier (by mass). Excess carrier protein is typical. For instance, the MENACTRA™ product has 16 μg saccharide (4 μg per serogroup) and 48 μg diphtheria toxoid, whereas the NIMENRIX™ product has 20 μg saccharide (5 μg per serogroup) and 44 μg tetanus toxoid.

Where a vaccine composition includes capsular saccharide from more than one serogroup, it is preferred that each separate conjugate uses the same carrier protein. Thus the carrier protein for meningococcal saccharides can be CRM197 (as in the MENVEO™ product), Dt (as in the MENACTRA™ product), or Tt (as in the NIMENRIX™ product). In some embodiments, however, different serogroups can use different carriers e.g. at least one serogroup conjugated to CRM197, and at least one serogroup conjugated to Tt.

Pneumococcal Capsular Saccharide Conjugates

Streptococcus pneumoniae causes bacterial meningitis and existing vaccines are based on capsular saccharides. Thus vaccine compositions of the invention can include at least one pneumococcal capsular saccharide conjugated to a carrier protein.

The invention can include capsular saccharide from one or more different pneumococcal serotypes. Where a composition includes saccharide antigens from more than one serotype, these are preferably prepared separately, conjugated separately, and then combined. Methods for purifying pneumococcal capsular saccharides are known in the art (e.g. see reference 76) and vaccines based on purified saccharides from 23 different serotypes have been known for many years. Improvements to these methods have also been described e.g. for serotype 3 as described in reference 77, or for serotypes 1, 4, 5, 6A, 6B, 7F and 19A as described in reference 78.

Pneumococcal capsular saccharide(s) will typically be selected from the following serotypes: 1, 2, 3, 4, 5, 6A, 6B, 7F, 8, 9N, 9V, 10A, 11A, 12F, 14, 15B, 17F, 18C, 19A, 19F, 20, 22F, 23F and/or 33F. Thus, in total, a composition may include a capsular saccharide from 1, 2, 3, 4, 5, 6, 7, 8, 9, 10, 11, 12, 13, 14, 15, 16, 17, 18, 19, 20, 21, 22, 23 or more different serotypes. Compositions which include at least serotype 6B saccharide are useful.

A useful combination of serotypes is a 7-valent combination e.g. including capsular saccharide from each of serotypes 4, 6B, 9V, 14, 18C, 19F, and 23F. Another useful combination is a 9-valent combination e.g. including capsular saccharide from each of serotypes 1, 4, 5, 6B, 9V, 14, 18C, 19F and 23F. Another useful combination is a 10-valent combination e.g. including capsular saccharide from each of serotypes 1, 4, 5, 6B, 7F, 9V, 14, 18C, 19F and 23F. An 11-valent combination may further include saccharide from serotype 3. A 12-valent combination may add to the 10-valent mixture: serotypes 6A and 19A; 6A and 22F; 19A and 22F; 6A and 15B; 19A and 15B; or 22F and 15B. A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F; 6A and 19A, etc.

Thus a useful 13-valent combination includes capsular saccharide from serotypes 1, 3, 4, 5, 6A, 6B, 7F, 9V, 14, 18C, 19 (or 19A), 19F and 23F e.g. prepared as disclosed in references 79 to 82. One such combination includes serotype 6B saccharide at about 8 μg/ml and the other 12 saccharides at concentrations of about 4 μg/ml each. Another such combination includes serotype 6A and 6B saccharides at about 8 μg/ml each and the other 11 saccharides at about 4 μg/ml each.

Particularly useful carrier proteins for pneumococcal conjugate vaccines are CRM197, tetanus toxoid, diphtheria toxoid and H. influenzae protein D. CRM197 is used in PREVNAR™. A 13-valent mixture may use CRM197 as the carrier protein for each of the 13 conjugates, and CRM197 may be present at about 55-60 μg/ml.

Where a composition includes conjugates from more than one pneumococcal serotype, it is possible to use the same carrier protein for each separate conjugate, or to use different carrier proteins. In both cases, though, a mixture of different conjugates will usually be formed by preparing each serotype conjugate separately, and then mixing them to form a mixture of separate conjugates. Reference 83 describes potential advantages when using different carrier proteins in multivalent pneumococcal conjugate vaccines, but it is known from the PREVNAR™ products that the same carrier can be used for multiple different serotypes.

A pneumococcal saccharide may comprise a full-length intact saccharide as prepared from pneumococcus, and/or may comprise fragments of full-length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. Depolymerisation can be used in order to provide an optimum chain length for immunogenicity and/or to reduce chain length for physical manageability of the saccharides. Where more than one pneumococcal serotype is used then it is possible to use intact saccharides for each serotype, fragments for each serotype, or to use intact saccharides for some serotypes and fragments for other serotypes.

Where a composition includes saccharide from any of serotypes 4, 6B, 9V, 14, 19F and 23F, these saccharides are preferably intact. In contrast, where a composition includes saccharide from serotype 18C, this saccharide is preferably depolymerised.

A serotype 3 saccharide may also be depolymerised, For instance, a serotype 3 saccharide can be subjected to acid hydrolysis for depolymerisation [79] e.g. using acetic acid. The resulting fragments may then be oxidised for activation (e.g. periodate oxidation, maybe in the presence of bivalent cations e.g. with MgCl₂), conjugated to a carrier (e.g. CRM197) under reducing conditions (e.g. using sodium cyanoborohydride), and then (optionally) any unreacted aldehydes in the saccharide can be capped (e.g. using sodium borohydride) [79]. Conjugation may be performed on lyophilized material e.g. after co-lyophilizing activated saccharide and carrier.

A serotype 1 saccharide may be at least partially de-O-acetylated e.g. achieved by alkaline pH buffer treatment [80] such as by using a bicarbonate/carbonate buffer. Such (partially) de-O-acetylated saccharides can be oxidised for activation (e.g. periodate oxidation), conjugated to a carrier (e.g. CRM197) under reducing conditions (e.g. using sodium cyanoborohydride), and then (optionally) any unreacted aldehydes in the saccharide can be capped (e.g. using sodium borohydride) [80]. Conjugation may be performed on lyophilized material e.g. after co-lyophilizing activated saccharide and carrier.

A serotype 19A saccharide may be oxidised for activation (e.g. periodate oxidation), conjugated to a carrier (e.g. CRM197) in DMSO under reducing conditions, and then (optionally) any unreacted aldehydes in the saccharide can be capped (e.g. using sodium borohydride) [84]. Conjugation may be performed on lyophilized material e.g. after co-lyophilizing activated saccharide and carrier.

Pneumococcal conjugates can ideally elicit anticapsular antibodies that bind to the relevant saccharide e.g. elicit an anti-saccharide antibody level >0.20 μg/mL [85]. The antibodies may be evaluated by enzyme immunoassay (EIA) and/or measurement of opsonophagocytic activity (OPA). The EIA method has been extensively validated and there is a link between antibody concentration and vaccine efficacy.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where an antigen is described as being “adsorbed” to an adjuvant, it is preferred that at least 50% (by weight) of that antigen is adsorbed e.g. 50%, 60%, 70%, 80%, 90%, 95%, 98% or more. It is preferred that diphtheria toxoid and tetanus toxoid are both totally adsorbed i.e. none is detectable in supernatant. Total adsorption of HBsAg can be used.

Amounts of conjugates are generally given in terms of mass of saccharide (i.e. the dose of the conjugate (carrier+ saccharide) as a whole is higher than the stated dose) in order to avoid variation due to choice of carrier.

Where animal (and particularly bovine) materials are used in the culture of cells, they should be obtained from sources that are free from transmissible spongiform encephalopathies (TSEs), and in particular free from bovine spongiform encephalopathy (BSE).

### MODES FOR CARRYING OUT THE INVENTION

Tt-Conjugates for Protecting Against Tetanus

In order to evaluate if Tt in a conjugate can protect in vivo against a lethal challenge by tetanus toxin (in accordance with Ph. Eur. 2.7.8), un-primed mice were immunized with MENITORIX™ (a bivalent MenC/Hib conjugate in which both the polysaccharide components are conjugated to Tt).

According to its SmPC, MENITORIX™ contains about 17.5 μg Tt. Two groups of mice (8 animals each) received a portion of MENITORIX™ such that each mouse was subcutaneously immunized with 10.5 μg Tt (group 1) or 2.1 μg Tt (group 2). Four weeks after vaccination, mice were challenged with tetanus toxin and in group 1, all mice survived. In group 2, six of eight (80%) of mice survived. In positive control groups with the bivalent ‘Td-pur’ vaccine, 100% of mice survived, while in the control group all mice died.

Dt-Conjugates for Protecting Against Diphtheria

In order to evaluate if Dt in a conjugate can protect against a lethal challenge by diphtheria toxin, guinea pigs were immunized with MENACTRA™ (a quadrivalent meningococcal conjugate based on Dt, with a Dt concentration of ˜48 μg per 0.5 ml human dose). Five animals received twice a human dose with a vaccination interval of 14 days. Two weeks after the second immunization, the guinea pigs were challenged with diphtheria toxin. Four of five animals survived. In the positive control, all animals survived and none in the negative control group.

CRM197-Conjugates for Protecting Against Diphtheria

In order to evaluate if CRM197 conjugates can confer protection against a lethal challenge by diphtheria toxin, guinea pigs were immunized with MENVEO™ (a quadrivalent meningococcal ACWY conjugate vaccine with ˜40 μg CRM197 per dose). Two groups each of 10 animals were used. Group 1 was immunized once with about 20 μg of CRM197 and group received about 8 μg of CRM197. Upon challenge with diphtheria toxin, no animals neither in group 1 nor 2 survived, while in the positive control group with DTP vaccine, 100% of the animals survived.

To investigate whether an adjuvant could improve CRM197's protection, MENJUGATE™ was used (a monovalent meningococcal serogroup C vaccine based on CRM197 and containing 12.5-25 μg CRM197 per dose, with an aluminium hydroxide adjuvant). A group of 10 guinea pigs was immunized once with about 6 μg CRM197 per animal. No animal survived. All animals survived in the positive control group, but no animals survived in the negative control group.

To investigate whether a higher dose could improve CRM197's protection, a double dose was used. Two immunizations each with a full human dose containing ˜40 μg of CRM197 were used. The second dose was given 14 days after the first immunization, and lethal challenge was carried out 14 days thereafter. Of five guinea pigs in the group vaccinated twice with MENVEO™, all animals died, while in the positive control group all animals survived.

Experiment with Dt Carrier in SYNFLORIX™

The above experiments investigated conjugates of meningococcal polysaccharides, and it was shown that meningococcal polysaccharides coupled to Dt as carrier are protective against a lethal challenge by diphtheria toxin in guinea pigs. Further experiments looked whether a Dt carrier can also be protective using a saccharide from another bacterium, for example Streptococcus pneumoniae. These experiments used SYNFLORIX™. This is a 10-valent pneumococcal conjugate vaccine in which the polysaccharides of 8 of the 10 serotypes (1, 4, 5, 6B, 7F, 9V, 14, and 23F) are all coupled to protein D derived from non-typeable H. influenzae and serotype 18C polysaccharide is conjugated to tetanus toxoid. Only the polysaccharide of serotype 19F is conjugated to Dt (3-6 μg Dt per vaccine dose). SYNFLORIX™ is adjuvanted with aluminum phosphate.

Five guinea pigs were each vaccinated once with a human vaccine dose of SYNFLORIX™ and a comparator group of five animals with MENJUGATE™ (each with one human vaccine dose). The five guinea pigs vaccinated with SYNFLORIX™ survived upon a subsequent challenge with diphtheria toxin while the five guinea pigs vaccinated with MENJUGATE™ died (as also seen in the previous experiment noted above).

This experiment confirms that the protective immunity of Dt used as a carrier of conjugate vaccines is independent of the nature of the polysaccharide source and its protective potency is not negatively impacted if conjugated to meningococcal and pneumococcal polysaccharides.

### SUMMARY

The experimental results are summarized as follows:

| Bacterial | Trade | Carrier | | Number | Adjuvant | Survival |
|---|---|---|---|---|---|---|
| saccharides | name | type | Carrier (μg) | of immunis^{ns} | (+/−) | rate |
| MenC/Hib | Menitorix | Tt | 17.5 | | − | 8/8 |
| MenACWY | Menactra | Dt | 48 | 2 | − | 4/5 |
| MenACWY | Menactra | Dt | 48 | 1 | − | 4/5 |
| Pnc 19F | Synflorix | Dt | 5 | 1 | + | 5/5 |
| MenACWY | Menveo | CRM197 | 44 | 2 | − | 0/5 |
| MenACWY | Menveo | CRM197 | 8 | 1 | − | 0/5 |
| MenC | Menjugate | CRM197 | 10 | 1 | + | 0/5 |
| MenC | Menjugate | CRM197 | 3 | 1 | + | 0/5 |

### CONCLUSIONS

Even when they are present only as the carrier protein in conjugate vaccines, Dt and Tt could confer protection against lethal challenge by diphtheria toxin or tetanus toxin, whereas the CRM197 mutant was not protective.

Reference 1 reported that Tt and Dt as carrier proteins in pneumococcal conjugate vaccine could protect against a lethal challenge with tetanus toxin or diphtheria toxin, and the author asserted in paragraph [0041] that the same effect would be seen with CRM197. The present inventor has shown that CRM197 surprisingly is a weaker immunogen compared to Dt as part of a conjugate vaccine, although CRM197-based conjugates are nevertheless effective vaccines to protect against the bacterial disease specified by the linked saccharide.

Thus, when developing combination vaccines which go beyond the existing hexavalent vaccine (D+T+Pa+Hib+HBsAg+IPV) it can be possible to add a meningococcal conjugate like MenC-Tt or MenC-Dt and remove the existing Dt or Tt component, but MenC-CRM197 could not be used in this way. Thus a useful combination vaccine would be D+Pa+Hib+HBsAg+IPV+MenC-Tt or T+Pa+Hib+HBV+IPV+MenC-Dt.

If Tt is used as the carrier for Hib, and Dt is used for MenC (and optionally for further meningococcal serogroups), both the Tt and Dt components can be removed, to give Pa+Hib-Tt+HBV+IPV+MenC-Dt, thereby reducing the antigenic complexity without reducing the breadth of protection. The combination includes five components (if Pa is considered as a single component) but has the same disease coverage as a 7-valent vaccine. There is still room for one further valence without becoming more complex than currently-marketed 6-valent vaccines.

Conversely, the fact that CRM197 is a weaker immunogen than Dt in the context of a conjugate vaccine makes this protein an attractive carrier when a conjugate vaccine is given concomitantly with current infant combination vaccines, because there may be lower potential for negative interference induced by the carrier protein. Thus, from the available MenC conjugate vaccines, MENJUGATE™ and MENINGITEC™ would be preferred over NEISVAC-C™ (which has a Tt carrier) when used in conjunction with current pediatric vaccines. Similarly, from the available MenACWY conjugate vaccines, MENVEO™ would be preferred over NIMENRIX™ (which has a Tt carrier) and MENACTRA™ (Dt carrier) when used in conjunction with current pediatric vaccines. Furthermore, from the available multivalent pneumococcal conjugate vaccines, PREVNAR™ and PREVNAR13™ would be preferred over SYNFLORIX™ (which includes Dt and Tt carriers) when used in conjunction with current pediatric vaccines.

It will be understood that the invention has been described by way of example only and modifications may be made whilst remaining within the scope and spirit of the invention.

### REFERENCES

[1] US2003/0099672.
[2] Knuf et al. (2010) Vaccine 28:744-53.
[3] Podda et al. (1991) Ann. Ig. 3:79-84.
[4] Vaccines. (eds. Plotkin & Orenstein). 4th edition, 2004, ISBN: 0-7216-9688-0.
[5] Micoli et al. (2011) Vaccine 29:712-20.
[6] Rondini et al. (2011) Clin Vaccine Immunol 18: 460-8.
[7] Vaccines (Plotkin, Orenstein, Offit) Fifth edition, 2008. ISBN 978-1-4160-3611-1.
[8] Monath et al. (2011) N Engl J Med 364:1326-33.
[9] Vaccine Design: The Subunit and Adjuvant Approach (eds. Powell & Newman) Plenum Press 1995 (ISBN 0-306-44867-X).
[10] Vaccine Adjuvants: Preparation Methods and Research Protocols (Volume 42 of Methods in Molecular Medicine series). ISBN: 1-59259-083-7. Ed. O'Hagan.
[11] Clausi et al. (2008) J Pharm Sci DOI 10.1002/jps.21390.
[12] WO2011/067669.
[13] WO2011/067672.
[14] WO2011/067673.
[15] WO2008/056263.
[16] WO2011/154442.
[17] WO2011/154443.
[18] WO2011/154444.
[19] WO90/14837.
[20] Podda & Del Giudice (2003) Expert Rev Vaccines 2:197-203.
[21] Podda (2001) Vaccine 19: 2673-2680.
[22] WO2008/043774.
[23] WO2005/097181.
[24] WO95/11700.
[25] US-2007/014805.
[26] WO2007/080308.
[27] WO2010/023551
[28] Brito et al. (2011) Vaccine 29:6262-6268.
[29] U.S. Pat. No. 8,092,813.
[30] WO2011/141819.
[31] U.S. Pat. No. 6,630,161.
[32] Nony et al. (2001) Vaccine 27:3645-51.
[33] WO01/41800.
[34] Module 1 of WHO's The immunological basis for immunization series (Galazka)
[35] Lyng (1990) Biologicals 18:11-17
[36] NIBSC code: 69/017
[37] NIBSC code: DIFT
[38] Sesardic et al. (2001) Biologicals 29:107-22
[39] NIBSC code: 98/560
[40] NIBSC code: TEFT
[41] Sesardic et al. (2002) Biologicals 30:49-68
[42] NIBSC code: 98/552
[43] Rappuoli et al. (1991) TIBTECH 9:232-238.
[44] Nencioni et al. (1991) Infect Immun. 59(2): 625-30.
[45] Denoel et al. (2002) Vaccine 20:2551-5.
[46] Liao et al. (2012) J Infect Dis. 205:237-43.
[47] Verdijk et al. (2011) Expert Rev Vaccines. 10:635-44.
[48] Module 6 of WHO's The immunological basis for immunization series (Robertson)
[49] Vanlandschoot et al. (2005) J Gen Virol 86:323-31.
[50] WO2007/054820.
[51] WO03/066094.
[52] Ramsay et al. (2001) Lancet 357(9251):195-196.
[53] Lindberg (1999) Vaccine 17 Suppl 2:S28-36.
[54] Buttery & Moxon (2000) J R Coll Physicians Lond 34:163-168.
[55] Ahmad & Chapnick (1999) Infect Dis Clin North Am 13:113-133, vii.
[56] Goldblatt (1998) J. Med. Microbiol. 47:563-567.
[57] European patent 0477508.
[58] U.S. Pat. No. 5,306,492.
[59] WO98/42721.
[60] Conjugate Vaccines (eds. Cruse et al.) ISBN 3805549326, particularly vol. 10:48-114.
[61] Hermanson (1996) Bioconjugate Techniques ISBN: 0123423368 or 012342335X.
[62] WO96/40242.
[63] W.H.O. Tech. Rep. Ser. 594:51, 1976.
[64] WO03/080678.
[65] Glode et al. (1979) J Infect Dis 139:52-56
[66] WO94/05325; U.S. Pat. No. 5,425,946.
[67] Arakere & Frasch (1991) Infect. Immun. 59:4349-4356.
[68] Michon et al. (2000) Dev. Biol. 103:151-160.
[69] Rubinstein & Stein (1998) J. Immunol. 141:4357-4362.
[70] WO2005/033148
[71] WO2005/000347.
[72] WO02/058737.
[73] WO03/007985.
[74] WO2007/000314.
[75] WO2007/000322.
[76] WHO Technical Report Series No. 927, 2005. Pages 64-98.
[77] US-2008/0102498.
[78] US-2006/0228381.
[79] US-2007/0231340.
[80] US-2007/0184072.
[81] US-2006/0228380.
[82] WO2008/143709.
[83] WO2007/071707
[84] US-2007/0184071.
[85] Jodar et al. (2003) Vaccine 21:3265-72.

1-5. (canceled)

6. A method for immunising an infant against meningococcal disease and tetanus, comprising a step of administering a vaccine containing a meningococcal capsular saccharide conjugated to a tetanus toxoid carrier, without administering unconjugated tetanus toxoid.

7. A method for immunising an infant against meningococcal disease and diphtheria, comprising a step of administering a vaccine containing a meningococcal capsular saccharide conjugated to a diphtheria toxoid carrier, without administering unconjugated diphtheria toxoid.

8. (canceled)

9. A combination vaccine comprising: (a) unconjugated diphtheria toxoid, and a saccharide conjugated to a tetanus toxoid carrier, but being free from unconjugated tetanus toxoid; or (b) unconjugated tetanus toxoid, and a saccharide conjugated to a diphtheria toxoid carrier, but being free from unconjugated diphtheria toxoid.

10. (canceled)

11. A combination vaccine comprising a saccharide conjugated to a tetanus toxoid carrier, and a saccharide conjugated to a diphtheria toxoid carrier, but being free from unconjugated tetanus toxoid and free from unconjugated diphtheria toxoid.

12. The vaccine of claim 9, wherein the vaccine comprises (i) a conjugate of a meningococcal capsular saccharide to tetanus toxoid, (ii) a conjugate of a Hib capsular polysaccharide to tetanus toxoid, and/or (iii) a conjugate of a pneumococcal capsular saccharide to tetanus toxoid.

13. The vaccine of claim 12, wherein the vaccine comprises capsular saccharide from meningococcal serogroup C conjugated to tetanus toxoid.

14. The vaccine of claim 13, wherein the vaccine comprises capsular saccharide from meningococcal serogroups C and Y, each conjugated to tetanus toxoid.

15. The vaccine of claim 14, wherein the vaccine comprises capsular saccharide from meningococcal serogroups A, C, W135 and Y, each conjugated to tetanus toxoid.

16. The vaccine of claim 12, further comprising 2, 3, or 4 of: unconjugated diphtheria toxoid; acellular pertussis antigen(s); inactivated poliovirus; and/or hepatitis B virus surface antigen.

17. The vaccine of claim 9, wherein the vaccine comprises (i) a conjugate of a meningococcal capsular saccharide to diphtheria toxoid, (ii) a conjugate of a Hib capsular polysaccharide to diphtheria toxoid, and/or (iii) a conjugate of a pneumococcal capsular saccharide to diphtheria toxoid.

18. The vaccine of claim 17, wherein the vaccine comprises capsular saccharide from meningococcal serogroup C conjugated to diphtheria toxoid.

19. The vaccine of claim 18, wherein the vaccine comprises capsular saccharide from meningococcal serogroups A, C, W135 and Y, each conjugated to diphtheria toxoid.

20. The vaccine of claim 17, further comprising 2, 3, or 4 of: unconjugated tetanus toxoid; acellular pertussis antigen(s); inactivated poliovirus; and/or hepatitis B virus surface antigen.

21. The vaccine of claim 11, wherein the vaccine comprises (i) a conjugate of a meningococcal capsular saccharide to tetanus toxoid, (ii) a conjugate of a Hib capsular polysaccharide to tetanus toxoid, (iii) a conjugate of a pneumococcal capsular saccharide to tetanus toxoid, (iv) a conjugate of a meningococcal capsular saccharide to diphtheria toxoid, (v) a conjugate of a Hib capsular polysaccharide to diphtheria toxoid, and/or (vi) a conjugate of a pneumococcal capsular saccharide to diphtheria toxoid.

22. The vaccine of claim 21, wherein the vaccine comprises a Hib capsular saccharide conjugated to tetanus toxoid, and a meningococcal capsular saccharide conjugated to diphtheria toxoid.

23. The vaccine of claim 22, wherein the vaccine comprises: a Hib capsular saccharide conjugated to tetanus toxoid; and capsular saccharide from meningococcal serogroups A, C, W135 and Y, each conjugated to diphtheria toxoid.

24. The vaccine of claim 22, wherein the vaccine comprises: a Hib capsular saccharide conjugated to diphtheria toxoid; and capsular saccharide from meningococcal serogroups A, C, W135 and Y, each conjugated to tetanus toxoid.

25. The vaccine of claim 21, further comprising 1, 2 or 3 of: acellular pertussis antigen(s); inactivated poliovirus; and/or hepatitis B virus surface antigen.

26. The vaccine of claim 9, including at least one aluminium salt adjuvant(s)

27. A kit comprising at least two kit components which, when mixed, result in the combination vaccine of claim 9.

28. (canceled)

29. A method for immunising an infant against multiple pathogens, comprising a step of immunising the infant with the combination vaccine of claim 9.

30. A method for immunising an infant against multiple pathogens, comprising a step of immunising the infant with the combination vaccine of claim 11.

31. The method of claim 29, wherein the infant is immunologically naïve to tetanus toxoid (Tt) and/or diphtheria toxoid (Dt) at the time of immunisation
